(19) United States
(12) Patent Application Publication
(10) Pub. No.: US 2016/0102067 A1
(43) Pub. Date: Apr. 14, 2016

(54) OXIRAN AMINES

(71) Applicant: V-BIOTEK HOLDING APS, Copenhagen K (DK)

(72) Inventor: Jens Steen OLSEN, Havdrup (DK)

(73) Assignee: V-BIOTEK HOLDING APS, Copenhagen K (DK)

(21) Appl. No.: 14/442,229

(22) PCT Filed: Nov. 15, 2013
(86) PCT No.: PCT/DK13/50383

§ 371 Date: May 12, 2015

(30) Foreign Application Priority Data
Nov. 15, 2012 (DK) PCT/DK2012/050419

(51) Int. Cl. C07D 303/48

(52) U.S. Cl. CPC C07D 303/48

(57) ABSTRACT

Disclosed herein are oxiran amines useful for the treatment of a variety of diseases. The oxiran amines are useful in the manufacture of pharmaceutical compositions. The pharmaceutical composition may be used for the treatment or prevention of a disease caused by a virus having a lipid membrane or the pharmaceutical composition may be used for diseases requiring cell proliferation or immune-regulation.

### INTRODUCTION

The present invention relates to novel oxiran mides, which may be used for a number of purposes including treatment of diseases caused by viruses, paracits, and bacteria. The compounds disclosed herein also find use in the treatment of diseases alleviated by proliferative action. Examples include diseases in the mouth cavity and burns. The novel alkamides have been isolated from the plant Trigonella foenum-graecum.

### BACKGROUND ART

Trigonella foenum-graecum (also termed Fenugreek or TFG herein) is an annual herb belonging to the legume family. TFG seed is a major constituent of curry and a part of traditional Indian and Asian cooking. The TFG seed are rich in phytochemicals, including proteins, steroidal saponin, flavanoids, tannic acids, stearic acid, vegetal oils, alkaloide trigonelline and 4-hydroxyisoleucine (Duke, 2001;Skaltsa, 2002).

Folkloric tales and ancient and traditional medicine has described many uses for TFG seeds and TFG extracts, including lactation stimulation, condiment, aid of labor, indigestion, improvement of general health, and improve metabolism (Basch et al., 2003;Ulbricht et al., 2007). In vitro studies have shown that a TFG seed extract may both induce apoptosis and cell death and have protective effects. TFG extracts protects Chang liver cells against ethanol-mediated toxicity (Kaviarasan et al., 2006) but TFG extracts may also induce apoptosis and cell death in the cell line H-60, primarily via steroid components (Hibasami et al., 2003). Anti-microbial activity of TFG extracts has been reported. Extracts from TFG sprouts has been shown to have in vitro anti-bacterial affect against the stomach bacteria helicobacter pylori (Randhir et al., 2004;Randhir & Shetty, 2007). However, to date no reports have shown antiviral effects of TFG extracts.

Both HSV-2 and HIV-1 establish life-long latent infections and for neither virus an effective vaccine exists nor has any cure have been developed. Worldwide an estimated 33 million people are infected with HIV-1 and more than 500 million people are infected with HSV-2 (Looker et al., 2008; UNAIDS, 2010). HIV-1 infection ultimately leads to acquired immune deficiency syndrome (AIDS) characterize by deteriorating immune response, attacks of opportunistic infections collectively leading to death.

HSV-2 is a very common and important human pathogen, causing localized infections of the genital mucosa but HSV-2 may also infect the skin and the pharynx. Normally the infection with HSV is benign and self-liming. However, in immune-compromised patients, such as HIV patients, transplantation patients and in neonates the infections may produce severe infections in the central nervous system, including acute necrotizing encephalitis and meningitis (Roizman et al., 2007). Furthermore, HSV-2 is an important co-factor for HIV-1 infection and thus inhibition of HSV-2 infection may possibly reduce spread of HIV-1 (Freeman et al., 2006;Rebbapragada et al., 2007). Development of dual-acting compounds and prophylactic drugs against HIV-1 and HSV-2 may therefore be an important goal to inhibit spread of the viruses.

The alkamide compounds of the present invention are derived from extracts of TFG. These compounds have for the first time been isolated and characterised herein.

The present invention is directed to the object of providing novel compounds for the treatment of various diseases, including virus related diseases, diseases which can be cured or alleviated by cell proliferation, and diseases which can be treated by immune-modulation. A need still exist for providing new compounds for treating such diseases.

### SHORT DESCRIPTION OF THE INVENTION

The present invention relates to novel alkamide compounds of the following general formulae:

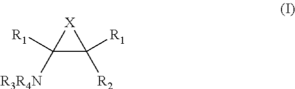

wherein
   X represents O or S,
   R₁ independently represents hydrogen; a straight or branched alkyl, alkenyl or alkynyl group containing up to 6 carbon atoms, optionally substituted by one or more halogen atoms or one or more groups R⁵; or a cycloalkyl or cycloalken group containing from 3 to 7 carbon atoms, said group optionally being substituted by one or more groups R⁵ or one or more halogen atoms,
   R₂ represents a straight or branched alkyl, alkenyl or alkynyl group containing 3 to 24 carbon atoms, said group optionally being substituted by one or more halogen atoms, a cycloalkyl group containing from 3 to 6 carbon atoms, or one or more groups R⁵,
   R₃ and R₄ may independently represent hydrogen, a straight or branched alkyl, alkenyl or alkynyl group containing up to six carbon atoms, said group optionally being substituted by one or more halogen atoms, or may together with the nitrogen atom to which they are joined or together with R₁ form 5 to 7 membered saturated or unsaturated heterocyclic ring containing up to three ring heteroatoms selected from nitrogen, oxygen and sulfur, which ring is optionally substituted by one or more groups selected from halogen, nitro, —S(O)pR⁶, C₁₋₄ alkyl, C₁₋₄ alkoxy, C₁₋₄ haloalkyl, C₁₋₄ haloalkoxy, ═O, and ═NO-R⁵, it being understood that a sulphur atom, where present in the ring, may be in the form of a group —SO₂— or —SO—;
   R₅ represents a straight or branched alkyl group containing up to six carbon atoms, said group optionally being substituted by one or more halogen atoms, a C₁₋₄ alkoxy, or a straight or branched alkenyl or alkynyl group containing from 2 to 6 carbon atoms, said group optionally being substituted by one or more halogen atoms; and
   R⁶ represents a straight or branched alkyl group containing up to six carbon atoms, said group optionally being substituted by one or more halogen atoms;
   p is 0, 1, or 3,
   and pharmaceutically acceptable salts thereof.

By the term ‘pharmaceutically acceptable salts’ is meant salts of the cations or anions which are known and accepted in the art for the formation of salts for therapeutical use. Suitable salts with bases include alkali metal (e. g. sodium and potassium), alkaline earth metal (e. g. calcium and magnesium), ammonium and amine (e.g. diethanolamine, triethanolamine, octylamine, morpholine and dioctylmethylamine) salts. Suitable acid addition salts, e. g. formed by compounds of formula (I) containing an amino group, include salts with inorganic acids, for example hydrochlorides, sulphates, phosphates and nitrates and salts with organic acids for example acetic acid.

Compounds of formulae (I) or (II) may exist in enolic tautomeric forms that may give rise to geometric isomers around the enolic double bond. Furthermore, in certain cases the above substituents may contribute to optical isomerism and/or stereoisomerism. All such forms and mixtures thereof are embraced by the present invention.

In the definitions of symbols in this specification including the accompanying claims unless otherwise specified, the following definitions generally apply to the radicals in the formulae (I): ‘halogen’ means a fluorine, chlorine, bromine or iodine atom; and ‘alkyl groups’ means straight- or branched-chain groups containing from 1 to 6 carbon atoms.

The heterocyclic ring may comprise a pyridine, pyrrole, imidazole, oxazibe, thiazine, pyrimidine, piperazine, aziridine, azirine, piperidine, diazirine, oxazoldine, imidazolidine, thiazolidine, isoxazolidine, pyrazolidine, isothiazolidine, oxazole, thiazole, isoxazole, pyrazole, isothiazole, morpholine, piperazine, thiazine, oxazine, pyrazine, pyridazine, diazetidine, azepine, azepane, triazine, tetrazine, triazine, tetrazine, triazine, adenine, guanine, thymine, cytosine, uracil, purine, pyrimidine, indole, benzimidazole, benzotriazole, or a quinoline group.

In a certain aspect of the invention R1 is hydrogen or a straight of branched alkyl having 1 to 3 carbon atoms. In a preferred aspect R1 is hydrogen.

R2 may in a certain aspect of the invention represent a straight or branched alkyl or alkenyl having up to 5 carbon atoms, such as between 5 and 20 carbon atoms. When R2 is an alkenyl 1 to 4 double bonds may be present and the double bond(s) may be in cis and trans configuration. The double bond may be positioned 3, 6, or 9 carbon atoms counted from the methyl terminal.

R3 and R4 are suitably hydrogen, or straights or branched alkyl having 1-3 carbon atoms. In a preferred aspect of the invention R3 and R4 are identical.

In a certain aspect of the invention, the compounds of formula (I) are preferably of the type that is represented by the following formula:

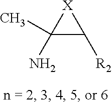

The compounds according to the general formulae (I) and (II) may be obtained in any suitable way. In a first aspect the compounds are obtained by chemical synthesis. In another aspect the chemical compounds are obtained from a natural source such as from fenugreek. When the compounds are obtained from fenugreek, an aqueous extract is generally obtained initially and the compounds of the invention are isolated from this extract.

The aqueous extract may be obtained by grinding the seeds, pouring hot or boiling water to the grinded seeds, and filtering off the solid particles. The extract may also be obtained by initially allowing the seeds to sprout by incubating the seeds in humid or aqueous conditions for 3 hours to 7 days. After the sprouting of the seeds they are treated with warm water having temperature of 70° C. or above, preferably boiling water. After filtering off of the solid parts a clear extract is obtained.

The extract may initially be treated with ethanol to precipitate the majority of the plant residues and polysaccharides from the extract. The precipitate may be removed by sedimentation or centrifugation. For easier storage, the solvent may be evaporated or otherwise removed so as to produce a powder. Alternatively the ethanol treated extract may be used directly in the subsequent process.

The powder is subsequently suspended in water and acidified to pH 1-4, preferably pH 2, with a strong acid such as hydrochloric acid. The acidified extract is extracted with a organic water immiscible solvent like heptane. After agitation the organic and the aqueous layer are separated and the aqueous layer is treated with an alkaline agent to obtain a pH above pH 9, preferably around pH 10. The alkaline aqueous phase is again extracted with an organic water immiscible solvent and agitated. A solid powder is obtained from the recovered organic phase by removing the solvent by evaporation, such as by evaporation under reduced pressure.

In an aspect of the invention the active compound (I) is prepared by chemical synthesis starting from commercial avaiable substances. In first step a fatty aldehyde or ketone compound may be reacted with a nitroalkyl to obtain a alkane chain substituted on adjacent carbons with an OH group and a NO₂ group. In a second step a double bond may be formed by elimination of the OH group. Optionally, the OH group is initially reacted with an acid, usually formic acid, acetic acid, or acetic acid anhydrate in present of an acid catalyst to form an ester befor the elimination reaction is performed. In a third step the oxiran ring is formed by reacting the double bond with a mixture of a peroxid, like H₂O₂ and a base, like NaOH. In a final step the nitro group is erduced to an amine by a suitable reduction agent such as NaBH₄. The reaction may optionally be performed in the presence of a catalyst, such as Co²⁺.

The compounds of the present invention may be used for treatment of a variety of diseases. In a first aspect the compounds of the invention may be used for curing diseases caused by viruses. It is presently believed that viruses having a lipid envelope membrane are especially susceptible to the compounds of the present invention. Examples of such viruses include herpes simplex virus (HSV), influenza virus, human papilloma virus (HPV) or human immunodeficiency virus (HIV).

In another aspect of the present invention the compounds may be used for treating diseases requiring a proliferation of cells. Such diseases include: wounds, such as a surgical wounds or burns, mouth cavity diseases, or periodontal diseases.

In a third aspect of the invention the compounds may be used for treating diseases caused by an immuno related defect. More specifically, the compounds of the present invention may be used for treating diseases influenced by interleukin-6, interleukin-10, CCL3 and interleukin-12. IL-6 is relevant to many diseases such as diabetes, artherosclerosis, depression, Alzheimers Disease, systemic lupus erythematosus, rheumatoid arthritis, autoimmune diseases, oral diseases, coronary disease, progression of infections by viruses, bacteria or protozoa, and hematological and solid malignancies. CCL3, which is also termed macrophage inflammatory protein (MIP)-1α, is the first of four members of the MIP-1 CC chemokine subfamily. CCL3 is able to attract monocytes/macrophages to sites of inflammation and may potentially inhibit the monocyte/macrophage uptake of HIV-1 via CCR5 ligation. It is therefore presently believed that the compounds disclosed herein may be applied in the treatment of various inflammation diseases, such as asthma, arthritis, or multiple sclerosis.

In a fourth aspect of the invention the compund of formula I may be used as anti-biotics. Especielly, the compounds of the invention have shown effects on MRSA (Methicillin-Resistant Staphylococcus Aureus) and MSSA (Methicillin-Sensitive Staphylococcus Aureus).

### SUMMARY OF THE FIGURES

FIG. 1: NMR data for compound isolated from extract with annotations.

FIG. 2: Antiviral effect of TFG extract. Vero cells were seeded and after overnight culture infected with HSV-2 strain MS pre-incubated for 30 min with TFG extract or PBS as control. After 24 h cells were fixed and stained and viral plaques were counted. All figures show mean+/−SD of three independent experiments.

FIG. 3: Tzmbl-HIV-1 reporter cells were seeded and after overnight culture infected with HIV-1 stains 89.6 or JR-CSF. Before infection the viruses were pre-incubated with TFG extract in the indicated concentrations for 60 min or with PBS as a control. All figures show mean+/−SD of two independent experiments.

FIG. 4: Antiviral effect of TFG extracts against Influenza A. MRC-5 fibroblast cells were seeded and after overnight culture infected with CMV stain AD169 treated with PBS (control, CTR) or TFG extract diluted either 100 or 30 times. After 3 days of infection the cells were fixed and stained for CMV protein accumulation. The number of infected cells was counted using fluorescence microscopy. The figures represent mean+/−SD of one of three independent experiments showing similar results.

FIG. 5: Antiviral effect of TFG extracts against human cytomegalovirus. MDCK cells were seeded and after overnight culture infected with Influenza A treated with PBS (control, CTR) or TFG extract diluted either 100 or 30 times. The number of infected cells was quantified using fluorescent staining. The figures represent mean+/−SD of one of two independent experiments showing similar results.

FIG. 6: Evaluation of TFG extract's toxicity and cell proliferative effect. Vero cells were treated with indicated concentrations of TFG extracts for 2 days and cell viability was evaluated using an MU assay.

FIG. 7: Evaluation of TFG extract's toxicity and cell proliferative effect human keratinocyte HaCaT cells were treated with indicated concentrations of TFG extracts for 2 days and cell viability was evaluated using an MIT assay.

FIG. 8: Human PBMCs were treated with TFG extract at the indicated concentrations and after two days of incubation the cell were assayed for viability using cell titre glow. The data depicted represents mean of 2 independent experiments+/−SD.

FIG. 9: Antiviral effect of heat-stable TFG extract is primarily via direct interaction with the virus. Vero cells were treated with TFG extracts (20 μg/ml) either before, simultaneously or after HSV-2 infection administered at time 0. After 24 h of virus infection, the cells were stained with crystal violet and subsequently virus plaques were counted.

FIG. 10: Vero cells were added TFG extract (20 μg/ml) or equivalent heat-treated TFG extract before adding HSV-2. After 24 h cells were stained and the number of plaques counted. The data in the figure represents the mean+/−SD of three individual experiments.

FIG. 11: Lipid addition inhibits TFG extract's antiviral effect. Per sample 30 μl of TFG extract (100 μg/ml) was incubated with lipofectamin2000 at the indicated volumes for 20 min. Immediately hereafter the extracts were incubated with HSV-2 for 30 min before infecting Vero cells. After 24 h the cells were stained and viral plaques enumerated. The data represents mean+/−SD of four independent experiments.

FIG. 12: TFG extract induces and augments secretion of pro-inflammatory IL-6 and CCL3. A to D) Freshly prepared PBMCs were stimulated with LPS (100 ng/ml), R848 (0.5 μg/ml) or TNF- [symbol] (25 ng/ml) or media as control in the absence of presence of TFG extract (10 μg/ml or 20 μg/ml). After 20 h the cell media was harvested. Levels of secreted IL-6 and CCL3 were measured using ELISA. E) Monocytic THP-1 cells were seeded and stimulated with TNF- [symbol] (25 ng/ml) or media as control in the absence of presence of TFG extract (20 μg/ml or 200 μg/ml). The cell media was harvested after 20 min. The level of secreted CCL3 was measured using ELISA. The data depicted represents mean+/−SD of four to six donors (A to D) or four independent experiments (E).

FIG. 13: Effect on IL-10 and IL-12 by TFG extracts in human primary cells. Freshly prepared PBMCs were stimulated with LPS (100 ng/ml), R848 (0.5 μg/ml) or TNF- [symbol] (25 ng/ml) or media as control in the absence of presence of TFG extract (10 μg/ml or 20 μg/ml). After 20 h the cell media was harvested. Levels of secreted IL-10 (A to C) and IL-12 (D and E) were measured using ELISA. The data depicted represents mean+/−SD of six donors (A to C) or four donors (D and E).

FIG. 14: In vivo effect of TFG extract by vaginal HSV-2 infection. Mice were treated with a gel containing 0.5 mg/ml TFG extract (A) or containing 2.5 mg/ml TFG extract (B). TFG extract was applied 12 h before and 12 h after vaginal challenge with HSV-2 (strain 333, 6.67×104 pfu/mouse). Every day after infection the disease severity was scored using a standard scoring system. The data depicted represents mean+/−SD of two separate experiments for A and one experiment for B.

FIG. 15: Growth rate of P. Falciparum in the presence of 2-methyl-3-nonyloxiran-2-amine or DMSO. The amount of incorporated [3H]-hypoxanthine was measured after 48 hours to correlate to the number of parasitized erythrocytes.

### DETAILED DESCRIPTION

The compounds described herein may be used for the treatment of various virus related diseases. Viral infection refers to an infection caused by a virus. Unlike bacteria viral replication is dependent on a host cell employing the host systems such as the transcription factor and translational machinery. The most common human diseases caused by viruses include common cold, the flu, cold sores, and warts.

In one embodiment according to the present invention a compound as described herein is used in the treatment of viral infections such as common cold, the flu, cold sores, and warts.

Specific examples of virus related diseases which may be treated with the compound described herein include herpes simplex virus (HSV). Herpes simplex virus 1 and 2 (HSV-1 and HSV-2) may be treated with the compound described herein, however, in a preferred aspect of the invention the disease is caused by HSV-2. Both HSV-1 (which produces most cold sores) and HSV-2 (which produces most genital herpes) are ubiquitous and contagious. They can be spread when an infected person is producing and shedding the virus.

Symptoms of herpes simplex virus infection include watery blisters in the skin or mucous membranes of the mouth, lips or genitals. Lesions heal with a scab characteristic of herpetic disease. Sometimes, the viruses cause very mild or atypical symptoms during outbreaks. However, as neurotropic and neuroinvasive viruses, HSV-1 and -2 persist in the body by becoming latent and hiding from the immune system in the cell bodies of neurons. After the initial or primary infection, some infected people experience sporadic episodes of viral reactivation or outbreaks. In an outbreak, the virus in a nerve cell becomes active and is transported via the neuron's axon to the skin, where virus replication and shedding occur and cause new sores.

The structure of herpes viruses consists of a relatively large double-stranded, linear DNA genome encased within an icosahedral protein cage called the capsid, which is wrapped in a lipid bilayer called the envelope. The envelope is joined to the capsid by means of a tegument. This complete particle is known as the virion. It is presently believed that the compound of the present invention exerts its action by interactions with the lipid bilayer.

HSV evades the immune system through interference with MHC class I presentation of antigen on the cell surface. It achieves this through blockade of the TAP transporter induced by the secretion of ICP-47[15] by HSV. TAP maintains the integrity of the MHC class I molecule before it is transported via the golgi apparatus for recognition by CD8+ CTLs on the cell surface.

Herpes viruses establish lifelong infections and the virus cannot currently be eradicated from the body. Treatment usually involves general-purpose antiviral drugs that interfere with viral replication, reducing the physical severity of outbreak-associated lesions and lowering the chance of transmission to others. Thus, the compound of the present invention clearly fulfil a need for the provision of a treatment method more efficient than, or at least an alternative to, the present general-purpose antiviral drugs.

Another disease which may be cured or alleviated by the present compound is influenza. Influenza, commonly known as the flu, is an infectious disease of birds and mammals caused by RNA viruses of the family Orthomyxoviridae, the influenza viruses. The term influenza includes disease caused by either influenza A, influenza B or influenza C virus. The most common symptoms are chills, fever, sore throat, muscle pains, headache (often severe), coughing, weakness/fatigue and general discomfort. Although it is often confused with other influenza-like illnesses, especially the common cold, influenza is a more severe disease caused by a different type of virus. Influenza may produce nausea and vomiting, particularly in children.

Typically, influenza is transmitted through the air by coughs or sneezes, creating aerosols containing the virus. Influenza can also be transmitted by direct contact with bird droppings or nasal secretions, or through contact with contaminated surfaces. Airborne aerosols have been thought to cause most infections, although, which means of transmission is most important is not absolutely clear.

The interior of the influenza virus particles the RNA genome is present and bound to the ribonuclear proteins. A capsid surrounds the genetic material and a lipid envelop is present outside the capsid. Present on the lipid envelope is various proteins including haemmagglutinin and ion channels. Presently it is assumed that the compounds of the present invention exert its action by interaction with the lipid membrane.

The compounds of the present invention may also be used to treat diseases caused by human papilloma virus (HPV). Warts are common benign epidermal lesions associated with human papillomavirus infection (HPV) infection. Warts referrers to a range of conditions, which differs in type of papillomavirus causing the conditions, the morphology, appearance on the body such as on the fingers, the foot, the face such as the lips or near the eyelids, or genital areas. Example of warts include common wart (verruca vulgaris) caused by HPV 1, 2, 4, 27, and 29, flat wart (verruca plana) caused by HPV 3, 10, 28, and 49, filiform or digitate wart, Palmar and plantar wart (verruca, verruca pedis) caused by HPV 1, mosaic wart, and genital wart (venereal wart, condyloma acuminatum, verruca acuminata).

Apart from being painful warts may also be a cosmetic problem there is no effective treatment of warts, which frequently reoccur a few months or years after the available treatment has been terminated.

In a preferred embodiment according to the invention the compound disclosed herein is used for the treatment of warts such as warts located on the fingers, the foot, the face such as the lips or near the eyelids, or genital areas.

In another aspect of the invention, a human immunodeficiency virus (HIV) related disease is treated with the compound disclosed herein. HIV is a lentivirus that causes acquired immunodeficiency syndrome (AIDS), a condition in humans in which progressive failure of the immune system allows life-threatening opportunistic infections and cancers to thrive. HIV includes several sub-types, including HIV-1 and HIV-2.

HIV infects vital cells in the human immune system such as helper T cells (specifically CD4+ T cells), macrophages, and dendritic cells. HIV infection leads to low levels of CD4+ T cells through three main mechanisms: First, direct viral killing of infected cells; second, increased rates of apoptosis in infected cells; and third, killing of infected CD4+ T cells by CD8 cytotoxic lymphocytes that recognize infected cells. When CD4+ T cell numbers decline below a critical level, cell-mediated immunity is lost, and the body becomes progressively more susceptible to opportunistic infections.

The HIV virus particle is roughly spherical with a diameter of about 120 nm, around 60 times smaller than a red blood cell, yet large for a virus. It is composed of two copies of positive single-stranded RNA that codes for the virus's nine genes enclosed by a conical capsid composed of 2,000 copies of the viral protein p24. The single-stranded RNA is tightly bound to nucleocapsid proteins, p7, and enzymes needed for the development of the virion such as reverse transcriptase, proteases, ribonuclease and integrase. A matrix composed of the viral protein p17 surrounds the capsid ensuring the integrity of the virion particle.

This is, in turn, surrounded by the viral envelope that is composed of two layers of fatty molecules called phospholipids taken from the membrane of a human cell when a newly formed virus particle buds from the cell. It is presently believed that the compounds of the present invention interact with the phospholopid bilayer to exert its action. The specific mode of action is not known presently to the inventors.

The present compounds may be use for treating a variety of diseases and disorders including diseases requiring a proliferation of cells. An example of a disease which be cured or the symptoms may be alleviated is periodontal diseases. Periodontitis (periodontosis, paradentosis, pyorrhea) is a dental disorder that results from progression of gingivitis, involving inflammation and infection of the ligaments and bones that support the teeth.

Left untreated for years it may results in loss of bone supporting the teeth and final loss of teeth. The conditions may involve one or more teeth.

Gingivitis is associated with little or no discomfort apart from redden, swollen and easily bleeding gums. Gingivitis is often caused by inadequate oral hygiene leaving the bacteria in plaque on the teeth causing the gums to become inflamed. Gingivitis is reversible with professional treatment and good oral home care. If gingivitis is left untreated plaque can spread and grow below the gum line and the condition may advance to periodontitis. Toxin released by bacteria in the plaque initiate an inflammatory response in the gums, which may become chronic and destroy the bone supporting the teeth. Gums separate from the teeth, forming pockets (spaces between the teeth and gums) that become infected. As the disease progresses, the pockets deepen and more gum tissue and bone are destroyed. Often, this destructive process has very mild symptoms. Eventually, teeth can become loose and may have to be removed.

Chronic periodontitis is recognized as the most frequently occurring form of periodontitis. Chronic periodontitis results in inflammation within the supporting tissues of the teeth, progressive attachment and bone loss and is characterized by pocket formation and/or recession of the gums (gingiva). It is prevalent in adults and a major cause of loss of teeth in adults, but the disease can occur at any age. Progression of attachment loss usually occurs slowly, but periods of rapid progression can occur.

Aggressive periodontitis is a condition that affects patient who are otherwise clinically healthy. Common features include rapid attachment loss and bone destruction and familial aggregation. Periodontititis, often with onset at a young age, associated with one of several systemic diseases, such as diabetes or osteoporosis (Periodontitis as a manifestation of systemic diseases). Necrotizing periodontal diseases is another form of infection characterized by necrosis of gingival tissues, periodontal ligament and alveolar bone. This condition is most often associated with systemic conditions including, but not limited to, HIV infection, malnutrition and immunosuppression.

Apart from is bacterial plaque other factors affecting the health of the gums include: Smoking, genetics, pregnancy, puberty, stress, medication, clenching/grinding of teeth, poor nutrition, diabetes and other systemic diseases.

Gingtivitis usually disappears with good self-care. In contrast, periodontitis requires repeat professional care. A person using good oral hygiene can clean only 2 to 3 millimetres ( 1/12 inch) below the gum line. A dentist can clean pockets up to 4 to 6 millimetres deep (⅕ inch) using scaling and root planning, which thoroughly remove tartar and the diseased root surface. For pockets of 5 millimetres (¼ inch) or more, surgery is often required. A dentist or periodontist may access the tooth below the gum line surgically (periodontal flap surgery) to thoroughly clean the teeth and correct bone defects caused by the infection. A dentist or periodontist may also remove part of the infected and separated gum (a gingivectomy) so that the rest of the gum can reattach tightly to the teeth and the person can then remove the plaque at home. A dentist may prescribe antibiotics (such as tetracyclines or metronidazole), especially if an abscess has developed. A dentist may also insert antibiotic-impregnated materials (filaments or gels) into deep gum pockets, so that high concentrations of the drug can reach the diseased area. Periodontal abscesses cause a burst of bone destruction, but immediate treatment with surgery and antibiotics may allow much of the damaged bone to grow back. If the mouth is sore after surgery, a chlorhexidine mouth rinse used for 1 minute twice a day may be temporarily substituted for brushing and flossing.

If a patient has 5 millimetres (¼ inch) or deeper pockets around most of their teeth, then they would then risk loss of all of their teeth over the years. If this not identified and the patient remains unaware of the progressive periodontal disease then, years later, they may be surprised that most of the teeth have suddenly seemed to become loose and that most or all of them may need to be extracted.

Pharmaceutical systemic treatment of gingivitis, periodontitis (aggressive and chronic), periodontitis as a manifestation of systemic diseases, and necrotizing periodontal diseases using tetracyclines is associated with a number of disadvantages the rapid emergence of tetracycline resistant bacterial strains and the occurrence of overgrowth of unsusceptible pathogens, such as Candida, during treatment. Short term treatment of periodontal infection with tetracyclines is often ineffective. Penicillins, which in general are highly effective antimicrobial compositions against anaerobic bacteria, have been shown to be ineffective against bacterial species important in peridental infections (e.g. P. gingivalis).

The limitations and disadvantages described above for the currently used surgical and non-surgical therapies reveal the unmet need for effective treatment of these dental conditions.

One highly preferred embodiment according to the present invention relates to the use of a compound as described herein for the treatment of a periodontal disease such as gingivitis, periodontitis (aggressive and chronic), periodontitis as a manifestation of systemic diseases, and necrotizing periodontal diseases.

Halitosis (or bad breath) is a very common temporary condition such as “morning breath”. Chronic halitosis, which is a more serious and persistent condition, is usually caused by persistent overpopulation of certain types of oral bacteria. Chronic halitosis is often associated with the periodontal diseases described herein.

In one embodiment according to the invention a compound as described herein is used for the treatment of halitosis. In a preferred embodiment said halitosis is chronic halitosis.

In another aspect of the invention the ability to proliferate cells is used to stimulate the treatment of wounds. The term “wound” refers to lesion of skin or mucosa (such as oral mucosa, gastric- and intestinal mucosa). The wound may be a result of an infection, injury, or surgery. Wound according to the invention also include chronic wounds and ulcers.

One preferred embodiment according to the invention relates to the use of a compound as described herein is used for the treatment of or preventing infection of a wound such as a surgical wound, a incised wounds, a penetration wound, a puncture wound, an abrasion, a chronic wound, or an ulcer.

Wounds may also results from bites. Human and mammal (mostly dog and cat, but also squirrel, gerbil, rabbit, guinea pig, and monkey) bites are common and occasionally cause significant morbidity and disability. The hands, extremities, and face are most frequently affected, although human bites can occasionally involve breasts and genitals. In addition to tissue trauma, infection from the biting organism's oral flora is a major concern.

In one embodiment according to the invention a compound as disclosed herein is used for the treatment of bites caused a human or a mammal, preferably a dog.

It has surprisingly turned out that wounds treated with the compounds according to the present invention heal faster. In addition scar formation is limited or absent. Scars are areas of fibrous tissue (fibrosis) that replace normal skin after injury and result from the biological process of wound repair in the skin and other tissues of the body. It is presently believed that the increased cell proliferation stimulated by the present compounds of the invention is the explanation for the observed faster healing.

According to an aspect of the invention the compounds disclosed herein may be used for treating diseases which are alleviated or cured by increased levels of pro-inflammatory IL-6 and CCL3.

Interleukin-6 (IL-6) is a pleomorphic cytokine involved in a number of physiologic and pathologic processes including response to trauma and infection as well as development and progression of inflammation and malignancy. IL-6 is relevant to many diseases such as diabetes (Kristiansen O P, Mandrup-Poulsen T (December 2005). “Interleukin-6 and diabetes: the good, the bad, or the indifferent?”. Diabetes 54 Suppl 2: S114-24.doi: 10.2337/diabetes. 54. suppl_2.S114. PM ID 16306329), artherosclerosis (Dubiński A, Zdrojewicz Z (April 2007). “[The role of interleukin-6 in development and progression of atherosclerosis]” (in Polish). Pol. Merkur. Lekarski 22 (130): 291-4.PMID 17684929), depression (Dowlati Y, Herrmann N, Swardfager W, Liu H, Sham L, Reim E K, Lanctot K L (March 2010). “A meta-analysis of cytokines in major depression”. Biological Psychiatry 67 (5): 446-457. doi:10.1016/j.biopsych.2009.09.033. PMID 20015486), Alzheimers Disease (Swardfager W, Lanctôt K, Rothenburg L, Wong A, Cappell J, Herrmann N (November 2010). “A meta-analysis of cytokines in Alzheimer's disease”. Biological Psychiatry 68(10): 930-941. doi:10.1016/j.biopsych.2010.06.012. PMID 20692646.), systemic lupus erythematosus (Tackey E, Lipsky P E, Illei G G (2004). “Rationale for interleukin-6 blockade in systemic lupus erythematosus”. Lupus 13 (5): 339-343.doi:10.1191/0961203304lu1023oa. PMC 2014821. PMID 15230289), rheumatoid arthritis (Nishimoto N (May 2006). “Interleukin-6 in rheumatoid arthritis”. Curr Opin Rheumatol18 (3): 277-281. doi:10.1097/01.bor.0000218949.19860.d1 . PMID 16582692), autoimmune diseases (Ishihara K, Hirano T. Cytokine Growth Factor Rev. 2002 August-October; 13(4-5):357-68. IL-6 in autoimmune disease and chronic inflammatory proliferative disease)), oral diseases (Nibali L, Fedele S, D'Aiuto F, Donos N Oral Dis. 2012 April; 18(3):236-43. doi: 10.1111/j.1601-0825.2011.01867.x. Epub 2011 Nov. 4. Interleukin-6 in oral diseases: a review), coronary diesease (Lim Nature Reviews Cardiology 9, 313 (June 2012) | doi:10.1038/nrcardio.2012.46 Coronary artery disease: IL-6 signaling linked with CHD) progression of infections by viruses, bacteria or protozoa and hematological and solid malignancies (Barton BE (August 2005). “Interleukin-6 and new strategies for the treatment of cancer, hyperproliferative diseases and paraneoplastic syndromes”. Expert Opin. Ther. Targets 9 (4): 737-752. doi:10.1517/14728222.9.4.737. PMID 16083340).

CCL3, which is also termed macrophage inflammatory protein (MIP)-1α, is the first of four members of the MIP-1 CC chemokine subfamily. CCL3 is able to attract monocytes/macrophages to sites of inflammation and may potentially inhibit the monocyte/macrophage uptake of HIV-1 via CCR5 ligation. It is therefore presently believed that the compounds disclosed herein may be applied in the treatment of various inflammation diseases, such as asthma, arthritis, or multiple sclerosis.

MIP-1 proteins mediate their biological effects by binding to cell surface CC chemokine receptors (3×104 to 5×105 receptors per cell), which belong to the G-protein-coupled receptor superfamily.

Receptor binding involves high affinity interactions and a subsequent cascade of intracellular events that rapidly leads to a wide range of target cell functions including chemotaxis, degranulation, phagocytosis, and mediator synthesis. Signal transduction events are initiated by the G-protein complex leading to its dissociation into Gα and Gβγ subunits.

MIP-1 family members orchestrate acute and chronic inflammatory host responses at sites of injury or infection mainly by recruiting proinflammatory cells. They are crucial for T-cell chemotaxis from the circulation to inflamed tissue and also play an important role in the regulation of transendothelial migration of monocytes, dendritic cells, and NK cells.

Thus, it is not surprising that MIP-1 proteins are key players in the pathogenesis of many inflammatory conditions and diseases including asthma, granuloma formation, wound healing, arthritis, multiple sclerosis, pneumonia, and psoriasis (Murdoch, C., & Finn, A. (2000). Chemokine receptors and their role in inflammation and infectious diseases. Blood, 95, 3032-3043). For example, CCL3 released from neutrophils that are recruited to sites of skin injury by mast cell-derived TNFα were found to be crucial mediators for macrophage influx in a murine model of cutaneous granuloma formation (von Stebut, E., Metz, M., Milon, G., Knop, J., & Maurer, M. (2003). Early macrophage influx to sites of cutaneous granuloma formation is dependent on MIP-1α/β released from neutrophils recruited by mast cell-derived TNFα. Blood, 101, 210-215). CCL3 also appears to be the critical macrophage chemoattractant in cutaneous wound repair, where it promotes healing (DiPietro, L. A., Burdick, M., Low, Q. E., Kunkel, S. L., & Strieter, R. M. (1998). MIP-1 alpha as a critical macrophage chemoattractant in murine wound repair. Journal of Clinical Investigation, 101, 1693-1698.), and it contributes to antigen-dependent basophil chemotaxis, histamine release and the development of eosinophilia in a model of allergic asthma (Venge, Lampinen, Hakansson, Rak, & Venge, 1996, Identification of IL-5 and RANTES as the major eosinophil hemoattractants in the asthmatic lung. Journal of Allergy and Clinical Immunology, 97, 1110-1115). MIP-1 proteins can also promote health by inducing inflammatory responses against infectious pathogens such as viruses, e.g. influenza (Menten, P., Wuyts, A., & von Damme, J. (2002). Macrophage inflammatory protein-1. Cytokine Growth Factor Reviews, 13, 455-481.) or parasites (Aliberti, J., Reis e Sousa, C., Schito, M., Hieny, S., Wells, T., Huffnagle, G. B., & Sher, A. (2000). CCR5 provides a signal for microbial induced production of IL-12 by CD8 alpha+ dendritic cells. Natural Immunology, 1, 83-87). For example, in Toxoplasma gondii infection CCL3 and CCL4 (and CCL5/RANTES) increase IL-12 release from dendritic cells by binding to CCR5, which results in enhanced Th1 immunity and clearance of the parasite (Venge et al., 1996). On the other hand, the MIP-1 receptors CCR3 and CCR5 promote HIV-1 infection as they are important co-receptors for M-tropic HIV-1 viruses on CD4+ target cells (Horuk, R. (2003). Development and evaluation of pharmaceutical agents targeting chemokine receptors. Methods, 29, 369-375).

The compounds of the present invention may in some instances be regarded dual or multiple acting drugs which may be used for simultaneous addressing the treatment of several diseases such as HIV-1 or HSV-2. Since both HIV-1 and HSV-2 are sexually transmitted, the compounds of the present invention may be mixed in a stable solution for topical application. One option is formulation in gels for skin application or as a microbiocide gel to be applied in the vagina or rectum. The latter solution may block or inactivate some sexually transmitted pathogens.

The compounds according to the invention are also suitable for the use in the treatment or prevention of malaria. Malaria is a mosquito-borne infectious disease caused protists of the genus Plasmodium. The present invention includes the treatment or prevention of the malaria disease caused by any species of Plasmodium, including P. falciparum, P. vivax, P. ovale, P. knowlesi and P. malariae. In a preferred aspect of the present invention the compounds according to the present invention is used or the prevention or treatment of malaria caused by P. falciparum. Among those infected, P. falciparum is the most common species identified (˜75%) followed by P. vivax (˜20%). P. falciparum accounts for the majority of deaths.

Species of Plasmodium have a certain life cycle which partly occurs in the human body after infection. The present invention includes treatment against species of Plasmodium in all its lifecycle stages, especially the lifecycle stages occurring in the body of a human. In the life cycle of Plasmodium, a female Anopheles mosquito (the definitive host) transmits a motile infective form (called the sporozoite) to a vertebrate host such as a human (the secondary host), thus acting as a transmission vector. A sporozoite travels through the blood vessels to liver cells (hepatocytes), where it asexually reproduces thousands of merozoites. These infect new red blood cells and initiate a series of asexual multiplication cycles that produce 8 to 24 new infective merozoites, at which point the cells burst and the infective cycle begins anew. In a process called gametocytogenesis, other merozoites develop into immature gametes, or gametocytes. When a fertilized mosquito bites an infected person, gametocytes are taken up with the blood and matured in the mosquito gut. The male and female gametocytes fuse and form zygotes (ookinetes), which develop into new sporozoites. The sporozoites migrate to the insect's salivary glands, ready to infect a new vertebrate host. The sporozoites are injected into the skin, alongside saliva, when the mosquito takes a subsequent blood meal. This type of transmission is occasionally referred to as anterior station transfer.

In an aspect of the invention the compounds according to the present invention may be used as antibaterial agents. It is expected that the compound has a general effect on various bacteria and therefore has a broad application. Thus, the compounds of the present invention may be used as a disinfectant, optinally after being suitably formulated. The disinfectant may be used for disinfecting various types of compartments including rooms in hospitals, such as surgeon rooms or operation theaters. Also domestic rooms may be cleaned with the disinfectant, including bath rooms. Other rooms which may be disinfected include stables for livestock such as pigs and cows, and laboratories. Tests have supported that the present compounds show good ablities of reducing the amount of certain types of Staphylococcus aureus, such as methicillin-resistant Staphylococcus aureus (MRSA), and methicillin-sensitive Staphylococcus aureus (MSSA).

MRSA is a bacterium responsible for several difficult-to-treat infections in humans. It is also called oxacillin-resistant Staphylococcus aureus (ORSA). MRSA is commenly used for any strain of Staphylococcus aureus that has developed, through the process of natural selection, resistance to beta-lactam antibiotics, which include the penicillins (methicillin, dicloxacillin, nafcillin, oxacillin, etc.) and the cephalosporins. Strains unable to resist these antibiotics are classified as methicillin-sensitive Staphylococcus aureus, or MSSA. MRSA is especially troublesome in hospitals, prisons and nursing homes, where patients with open wounds, invasive devices, and weakened immune systems are at greater risk of infection than the general public. The evolution of such resistance does not cause the organism to be more intrinsically virulent than strains of Staphylococcus aureus that have no antibiotic resistance, but resistance does make MRSA infection more difficult to treat with standard types of antibiotics and thus more dangerous. Thus, the present invention suggest a method for treatment of difficult-to-treat diseases like MRSA and MSSA by administering a compound according to the present invention to a patient suffering from infection or in risk of being infected with Staphylococcus auraus.

The compounds according to the present invention may be formulated in any pharmaceutical form and together with any appropriate pharmaceutically acceptable additive.

The pharmaceutical composition comprising a compound according to the invention may be formulated in a number of different manners, depending on the purpose of the particular medicament and the type of administration. It is well within the scope of a person skilled in the arts to formulate compositions that are in accordance with the preferred type of administration.

The medicament comprising the extract according to the invention may be prepared by any conventional technique, e.g. as described in Remington: The Science and Practice of Pharmacy 1995, edited by E. W. Martin, Mack Publishing Company, 19th edition, Easton, Pa.

The medicament may comprise pharmaceutical acceptable additives such as any conventionally used pharmaceutical acceptable additive, which should be selected according to the specific formulation, intended administration route etc. For example the pharmaceutical acceptable additives may be any of the additives mentioned in Nema et al, 1997. Furthermore, the pharmaceutical acceptable additive may be any accepted additive from FDA's “inactive ingredients list”, which for example is available on the internet address http://www.fda.gov/cder/drug/iig/default.htm.

One preferred embodiment of the present invention is to provide a pharmaceutical composition formulated for topical application on a local, superficial and restricted area such as the a wound, a cold sore, a wart, acne, diaper rash, rectum, genitals, etc.

In said above-mentioned embodiment, the medicament may be formulated as an ointment, a lotion, a crème, a bath admixture, a gel, a paste, a milk, a suspension, an aerosol, a spray, a film, a foam, a serum, a swab, a pledget, a pad, a patch, a powder, a paste, a liniment, viscous emulsion, porridge, or another formulation which is appropriate for topical administration.

Such compositions for topical administration may further include physiologically acceptable components such as carriers, surfactants, preservatives, stabilizing agents, buffers, excipients and emulsifiers suited for this type of administration. Suitable components for topical delivery systems are preferably chosen from components that do not cause excessive or unavoidable irritation or pain to the recipient. Carriers include diluents and provide the medium in which the pharmaceutical constituents are dissolved, dispersed or distributed.

The medicament according to the invention may comprise, but are not restricted, a carrier such as an aqueous liquid base, nonaqueous liquid base, water soluble gel, a mineral oil base, emulsion, ointment, crème, gel or lotion, suspension of solid particles in a liquid.

The topical availability of drugs depends on various factors including their ability to dissolve in the carrier (gel, cream-hydrophilic), and their ability to permeate the skin barrier (i.e., the stratum corneum-hydrophobic), thus requiring a unique hydrophobic-hydrophilic balance. Formulations may require addition of excipients, such as permeation enhancers and solubilizers to facilitate either or both of the transport processes (dissolution into vehicle and diffusion across skin). Additives, such as alcohols, fatty alcohols, fatty acids, mono- di- or tri-glycerides, glycerol monoethers, cyclodextrin and derivatives, polymers, bioadhesives, terpenes, chelating agents and surfactants have been disclosed to increase transdermal delivery of drugs. It is within the present invention to make use of such excipients.

Any method, not limited to the above-mentioned, for increasing transdermal delivery is within the scope of the present invention. The medicament according to the present invention may therefore comprise surfactants such as ionic and/or non-ionic surfactants. Suitable non-ionic surfactants include for example: fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronic); fatty acid alkylolamides (fatty acid amide polyethylene glycols); alkyl polyglycosides, N-alkyl-, N-alkoxypolyhydroxy fatty acid amide, in particular N-methyl-fatty acid glucamide, sucrose esters; sorbitol esters, esters of sorbitol polyglycol ethers and lecithin. Ionic surfactants include for example sodium lauryl sulfate, sodium laurate, polyoxyethylene-20-cetylether, Laureth-9, sodium dodecylsulfate (SDS) and dioctyl sodium sulfosuccinate.

Alcohols include, but are not limited to, ethanol, 2-propanol and polyols such as polyethylene glycol (PEG), propylene glycol, glycerol, propanediol.

Methods for enhancing drug delivery through topical administration may be applied with the present invention, and include any means of increasing absorption, minimizing metabolism, and/or prolonging the half-life of the active ingredient of the medicament such as the extract of Trigonella foenum-graecum. Such means include the use of transporters of the type liposomes, ISCOMs, nano-particles, microspheres, hydrogels, organogels, polymers or other micro-encapsulation techniques.

Medicament for topical delivery according to the present invention comprising may comprise any suitable amount of the compounds according to the invention, such as 0.01 to 50 wt %, preferably 0.1 to 30 wt % by weight.

Another preferred embodiment of the present invention is to provide a medicament formulated for oral administration such as a mouth wash.

In one preferred embodiment the medicament is formulated as a mouth wash such as by dissolving or disperging the compound according to the invention in a liquid.

The liquid may be any useful liquid, however it is frequently preferred that the liquid is an aqueous liquid. It is furthermore preferred that the liquid is sterile. Sterility may be conferred by any conventional method, for example filtration, irradiation or heating.

It is within the scope of the present invention to supply a medicament, and uses thereof, comprising a compound of the invenion for the treatment of clinical conditions described above involving an infection or an increased risk of acquiring an infection. For example, but not limited to, clinical conditions involving infection, or is at risk of being infected by a microbial species. In one embodiment of the invention the compound is co-administered with at least one second active ingredient. Preferably the compound of the invention and the second active ingredient are present in the same medicament. Alternatively, they may be supplied in a kit of parts. Preferably, said second active ingredient is an antimicrobial substance, for example an antiseptic, antibiotic, antifungal, antiparasitic or antiviral agent.

In an embodiment according to the present invention, the compound of the invention is a constituent in a tooth-paste.

According to the present invention the compound is present in “a pharmaceutical effective dosage” of the composition. A pharmaceutical effective dosage refers to the amount necessary to induce the desired biological effect on the subject in need of treatment.

The medicament according to the present invention may be administrated once or more than once a day, for example they may be administered in the range of 2 to 10 times a day, such as 2 to 7 times, for example 2 to 5 times, such as 2 to 4 times, such as 2 to 3 times a day.

The medicament according to the present invention may be administrated to the subject for a period of treatment of one or more than one week such as two weeks, three weeks, four weeks, five weeks, six weeks, seven weeks, eight weeks or more than eight weeks. The treatment may be repeated on subjects, who relapse.

### EXAMPLES

### Example 1

Preparation of an extract from Trigonella foenum-graecum seeds was performed as follows: 500g seeds of Trigonella foenum-graecum were soaked in 2.5 l water for approximately 24 hours. Following the pre-soaking the seeds were cooked for 20 minutes and remains of the seeds were removed from the mixture. The extract was chilled.

The extract was treated with about 800 ml ethanol to precipitate polysaccharides and plant residues. The mixture was subjected to centrifugation at 9000 rpm, the reminiscence was harvested, and the ethanol was evaporated together with some of the water. Subsequently, the extract was filtered through a cellulose filter (0.45 μm). This process produced a dry matter content of approx. 18 g/l. The aqueous extract was freeze dried and a powder was obtained.

5 ml of a powder dissolved in water at a concentration of 10 mg/ml was adjusted to pH 2 using 1M hydrochloric acid and mixed with 10 ml heptane. The mixture was agitated and the aqueous phase was isolated and adjusted to pH 10 using aqueous sodium carbonate. This aqueous layer was agitated with heptane (5 ml) and the organic phase was harvested. The organic phase was separated in two fractions and subjected to evaporation under nitrogen. One of the fractions was used for chemical analysis (example 2) and the other was used for biological assays (example 3).

### Example 2

Chemical analysis of vial from example 1:

LC MS Analysis

Summit 4 with MS detector

Positive ionization

Column: Primesep D

Eluent A: 0.1 M formic acid

Eluent B: Acetonitril

SCAN mode, 50-1000 amu

GC MS Analysis

Agilent GC with MSD detector

Column: f.eks. Zebron ZB-Wax (nr. 27)

SCAN mode 50-550 amu

A LC-MS and the GC MS analyses showed that several compounds having the same motif were present in the sample of the active fraction prepared in example 1. The molecules in the sample had the following composition:

C₁₈H₃₅NO

C₁₆H₃₃NO

C₁₄H₂₉NO

C₁₂H₂₉NO

C₁₀H₂₇NO

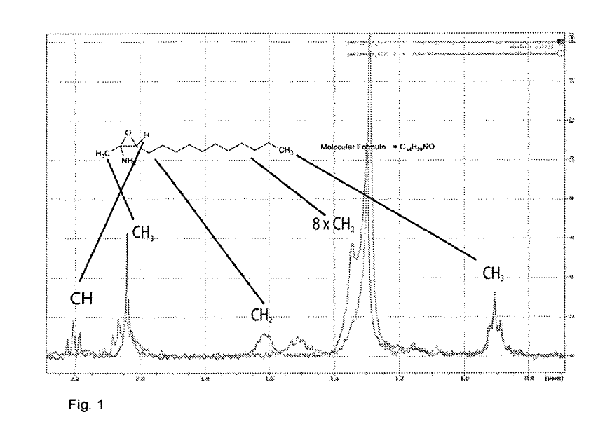

¹H NMR was prepared of a sample of fraction 1A in DMSO-d6 and Methanol-d6, respectively. A signal was detected at 6.88 ppm for the DMSO-d6 sample but disappeared in the Methanol-d6 sample, which is consistent with a NH₂ group. One of the plurality of compounds was selected for detailed analysis and FIG. 1 shows the correspondence between the structure of the identified compound and the ¹H NMR diagram.

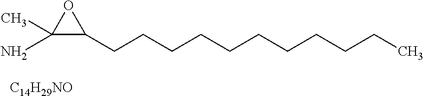 [SMILES: "C14H29NO" ; CCCCCCCCCCCC1OC1(C)N]

The 5 compounds identified in the LC-MS and the GC MS analyses can be represented by the chemical formulae below:

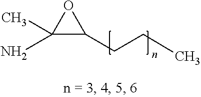

### Example 3

The vial from example 1 was tested for HSV-2 activity.

Vero kidney epithelial cells were grown in Dulbecco's Modified Essential Medium (DMEM) (Lonza, Basel, Switzerland) containing 10% heat-inactivated foetal calf serum (FCS) and 50 U/ml penicillin and 50 μg/ml streptomycin (lnvitrogen, Glostrup, Denmark). For virus plaque assays, Vero cells were seeded in 24-well plates at a density of 7-9×10⁵ cells per well to obtain 95% confluence after overnight culture. HSV-2 strains were amplified in Vero cells and quantified by viral titration as previously described (Ank et al., 2006). Twenty-four hr after transfection the cell media was renewed and 48 h post transfection, virus-containing supematant was harvested, filtered through a 0.45 μm filter and stored at −80° C.

A standard Vero cell plaque assay was used to evaluate the direct antiviral activity. The second vial from example 1 was reconstituted in water containing 0.005% formic acid and subsequent added 1/10 volume 10× PBS. 30 μl of the solution was mixed with 30 μl HSV-2 solution. The mixture was incubated in 30 min at room temperature. 50 μl of the incubated mixture was added to 95% confluent Vero cells. After 24 h of incubation, cells were fixed for 10 min using 4% formaldehyde (Polysciences, Eppelheim, Germany) in PBS and stained with 0.5% crystalviolet (Sigma-Aldrich, Copenhagen, Denmark) in PBS/10% EtOH after which viral plaques were enumerated.

The plaques was evaluated on a 4 stage scale (−, +, ++, +++). The vial containing the compound identified in example 2 showed full activity (+++). The activity decreased by dilution, indicating the existance of an S-shaped dosage-response curve. Combinations of the content of the vial from example 1 with other diluted fractions from the LC-MS fractionation revealed that the content of the vial from example 1 was needed for full activity.

### Example 4

Material and Methods

Cells. Vero kidney epithelial cells, human alveolar carcinoma epithelial A549 cells, human embryonic kidney (HEK)293T cells and human keratinocyte HaCaT cells were grown in Dulbecco's Modified Essential Medium (DMEM) (Lonza, Basel, Switzerland) containing 10% heat-inactivated foetal calf serum (FCS) and 50 U/ml penicillin and 50 μg/ml streptomycin (Invitrogen, Glostrup, Denmark). HEK293 cells stably expressing TLR4 were grown in DMEM containing 10% heat-inactivated foetal calf serum (FCS), 50 U/ml penicillin and 50 μg/ml streptomycin (Invitrogen, Glostrup, Denmark) and 500 μg/ml G418. Human monocytic THP-1 cells and human peripheral blood mononuclear cells (PBMCs) were cultured in RPMI1640 (Lonza, Basel, Switzerland) supplemented with 2 mM L-glutamine, 10 mM HEPES, 50 U/ml penicillin and 50 μg/ml streptomycin and 10% heat-inactivated FBS (Invitrogen, Glostrup, Denmark). For PBMC purification, leukocyte-enriched buffy coats were obtained from the Skejby Hospital Blood Bank or cells were purified from freshly drawn blood. PBMCs were purified by Isopaque-Ficoll separation and frozen down in RPMI1640 growth media containing 10% DMSO (Sigma-Aldrich, Copenhagen, Denmark) or used directly. Before experiments PBMCs were carefully thawed and for stimulation experiments and viability assays the PBMCs were seeded in 96-well culture plates at a density of 2×10⁵ cells per well and cultured overnight before further treatment. THP-1 cells were seeded at a density of 1×10⁵ cells per 96-well six h before further treatment. For viability assays, HaCaT and Vero cells were seeded at the density of 1×10⁴ cells per well. For virus plaque assays, Vero cells were seeded in 24-well plates at a density of 7-9×10⁵ cells per well to obtain 95% confluence after overnight culture.

Viruses. HSV-2 strains were amplified in Vero cells and quantified by viral titration as previously described (Ank et al., 2006). HIV-1 strains 89.6 and JR-CSF were produced in HEK293T cells. Briefly, HEK293T were seeded at 5×10⁴ per cm² and transfected with 10 μg HIV-1 plasmid per T80 bottle (Nunc, Roskilde, Denmark) using calcium phosphate precipitation. Plasmids for HIV-1 strains 89.6 and JR-CSF were obtained through the NIH AIDS Research and Reference Reagent Program, Germantown, Md., USA. Twenty-four hours after transfection the cell media was renewed and 48 hours post transfection, virus-containing supernatant was harvested, filtered through a 0.45 μm filter and stored at −80° C. Virus infectivity was determined on TZM-bl cells, as previously described (Kirkegaard et al., 2011).

MTT and cell titer glow cytotoxicity assays. To evaluate toxicity in adherent cells, cells were seeded in 96-well plates and maintained over night before applying TFG extract. After 48 h incubation, the cells were stained with the (3-(4,5-Dimethylthiazol-2-yl)-2,5-Diphenyltetrazolium Bromide)(MTT) substrate. Briefly, cells were incubated for 3 h in media that contained 0.5 mg/ml MTT (Sigma-Aldrich, Copenhagen, Denmark). Subsequently, cells were lysed with a 1:1 vol 96% EtOH and DMSO; cell survival was quantified by reading absorbance at 570 nm. For non-adherent THP-1 cells and PBMCs quantification of viability was performed using cell titre glow (CTG) (Promega, Nacka, Sweden). Twenty μl of THP-1 cell suspension was transferred to the white plate (Perkin-Elmer, Skovlunde, Denmark). Subsequently, 25 μl of CTG reagent was added, the plate was shaken and the level of luminescence signal was measured. For PBMCs, 50 μl of CTG reagent was added to 50 μl of media containing cells after which 50 μl of the solution was transferred to the white plate and left for 10 min to stabilize luminescent signals. Luciferase activity as a measure of viability was quantified using a Fluster Omega plate reader (BMG Lebech, Rotenberg, Germany).

HSV-2 plaque assay. A standard Vero cell plaque assay was used to evaluate the direct antiviral activity of the TFG seed extract. Ninety-five % confluent cells were either treated with the extracts before or after adding HSV-2 (stain MS) or TFG extract was pre-incubated for indicated time and concentrations before addition to the cells. For experiments with lipid addition, Lipofectamin2000 (Invitrogen, Glostrup, Denmark) was added at indicated concentrations to the mix of TFG extract and virus or virus alone for 20 min. Heat-treatment of TFG extracts was performed for 20 min at 56° C. The control cultures received PBS or virus was mixed with PBS. After 24 h of incubation, cells were fixed for 10 min using 4% formaldehyde (Polysciences, Eppelheim, Germany) in PBS and stained with 0.5% crystalviolet (Sigma-Aldrich, Copenhagen, Denmark) in PBS/10% EtOH after which viral plaques were enumerated.

TZM-bl HIV infectivity assay. Hela-derived TZM-bl cells were used to evaluate anti-HIV infectivity. TZM-bl cells express the HIV receptor CD4 and coreceptors CCR5 and CXCR4 and harbor a luciferase β-galactosidase reporter system under the control of the HIV-1 long terminal repeats (LTRs). TZM-bl cells were seeded in 96-well culture plates at a density of 1×10⁴ cells per well and cultured overnight. Cells were infected with HIV-1 strains 89.6 or JRCSF (TCID50 of 550). Before addition of virus to the cells, the virus was pre-incubated with indicated concentrations of TFG extract for 60 min at room temperature. After three days media was removed and cells were incubated with 90 μl 0.5% Nonidet P-40 in PBS for at least 45 min in order to inactivate the virus. Luciferase activity was measured using 90 μl of britelite plus reagent (Perking-Elmer, Skovlunde, Denmark) per well. After mixing, 150 μl of the solution was transferred to white 96-well plates (Perkin-Elmer, Skovlunde, Denmark). Luciferase activity was quantified using a FLUOstar Omega plate reader (BMG Labtech, Ortenberg, Germany).

CMV assay. Confluent MRC-5 cells were infected with CMV pre-incubated with TFG extract for 30 min or PBS as control. After three days of infection cells were washed with PBS and fixed and permabilized with 80% acetone for 10 minutes. After rinsing, the cells were incubated with 1:10-diluted monoclonal anti-CMV antibody (clone DDG9+CCH2, Dako, Glostrup, Denmark) for 30 min and subsequently incubated 30 min with FITC-conjugated goat anti-murine F(ab)2 antibody (Dako, Glostrup, Denmark). CMV-positive cells were visualized by fluorescence microscopy.

Influenza A virus assay. Viruses were incubated with TFG extract for 30 min at room temperature before adding to MDCK cells seeded in 96-well culture plates. After appropriate incubation time, the number of infected cells was visualized by fluorescent staining for viral proteins using IMAGEN kits (Oxoid, Thermo Fischer Scientific, Roskilde, Denmark) and the number of infected cells was counted using fluorescent microscopy.

Stimulation experiments. Cells were pre-treated with indicated concentrations of TFG extract for 30 min before stimulation with TLR4 ligand LPS (100 ng/ml, Sigma-Aldrich, Copenhagen, Denmark), TLR7/8 ligand R848 (0.5 μg/ml, InVivoGen, Toulouse, France) or TNF-α (25 ng/ml, R&D Systems, Abingdon, UK). After 20 h cell culture supernatants were harvested and stored at −80° C. until analysis by ELISA.

ELISA. Harvested cell culture supernatants were assayed using Duoset ELISA for CCL3/MIP-1α (R&D Systems, Abingdon, UK) or Cytoset ELISAs for IL-6, IL-10 and IL-12p40/p70 (Invitrogen, Glostrup, Denmark). ELISAs were performed as specified by the manufacturers.

Mice and TFG gel. The mice used in this study were 7 week-old, C57BL/6, females (Taconic M&B, Ry, Denmark). All animal experiments described were reviewed and approved by The Animal Experiments Inspectorate, Copenhagen, Denmark (approval number 2009/561/1641). The TFG extract gel with a final concentration of 0.5 and 2.5 mg/ml from 10 mg/ml TFG extract diluted in PBS and subsequently mixed with a hydroxyethylcellulose (HEC) gel solution (universal HEC placebo gel, NIH AIDS Research and Reference Reagent Program, Germantown, Md., USA). For the control group, we used the universal HEC placebo gel diluted in PBS. To synchronize the mice susceptibly to HSV-2 infection, mice were pre-treated with 200 μL of subcutaneously administered medroxyprogesterone diluted in PBS, at a concentration of 10 mg/ml, (Depo-Provera; Pfizer, Ballerup, Denmark) 5 days before the HSV-2 infection. The intravaginal infection was achieved with a lethal dose of strain 333 HSV-2 (6.67×10⁴ pfu/mouse), delivered in 20 μl of Iscoves medium (Lonza, Basel, Switzerland).

Mouse vaginal infection study. Mice were cages into two groups; one received 20 μl TFG seed extract vaginal gel, and the other received HEC placebo gel. The gel was applied 12 h before and 12 h after the HSV-2 infection. Mice were anesthetized with isoflurane (2-chloro-2-(difluoromethoxy)-1,1,1-trifluoro-ethane) for the gel applications and for the infection. To allow absorption of the gel or virus, mice remained anesthetized for 5-10 min after each application. The follow-up included daily monitoring of weight and disease score, based on the following scale: 0: healthy; 1: genital erythema; 2: moderate genital infection; 3: purulent genital lesions and/or in generally poor condition; 4: hind limb paralysis (leading to euthanasia).

Results

Anti-Viral Effect of TFG Seed Extracts Against HSV-2, HIV-1 and CMV

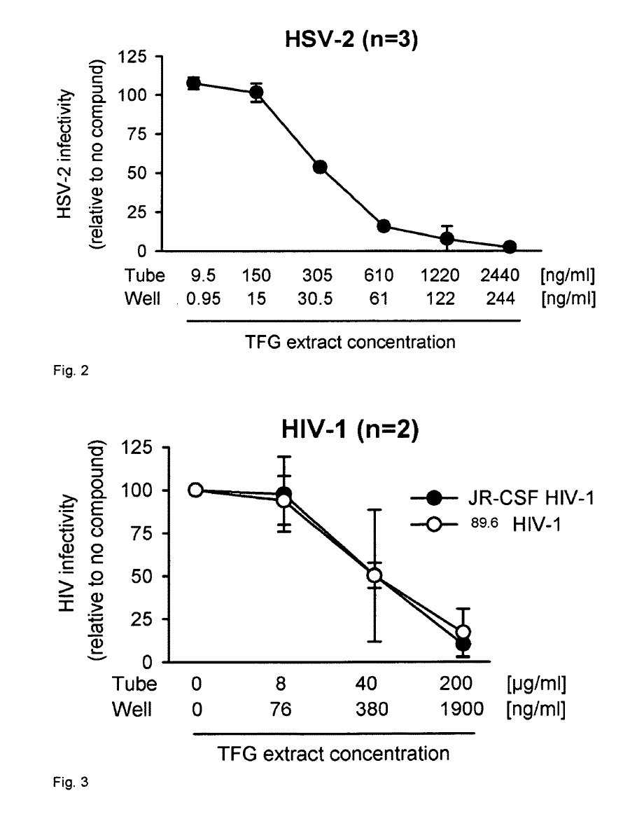

In order, to determine the direct antiviral effect of TFG extracts against HSV-2 and HIV-1, we pre-treated virus with extracts in decreasing concentrations and subsequently infected cells. After appropriate time of incubation the level of infection was quantified using plaque counts for HSV-2 and a luciferase reporter assay for HIV-1. TFG extract efficiently inhibits both HSV-2 (FIG. 2) and HIV-1 (FIG. 3). The 50% inhibitory concentration (IC₅₀) for HIV-1 was 40 μg/ml in the pre-incubation tubes and 380 ng/ml in the cell culture plates. The IC₅₀ for HSV-2 was much lower with an IC₅₀ of approximately 300 ng/ml in the pre-incubation tubes leading to a concentration of 30 ng/ml in the cell culture wells. In conclusion, TFG extracts efficiently inhibits virus infections with the major human pathogens HIV-1 and HSV-2.

TFG Extract Selective Effects Cell Proliferation and Evaluation of Cell Toxicity

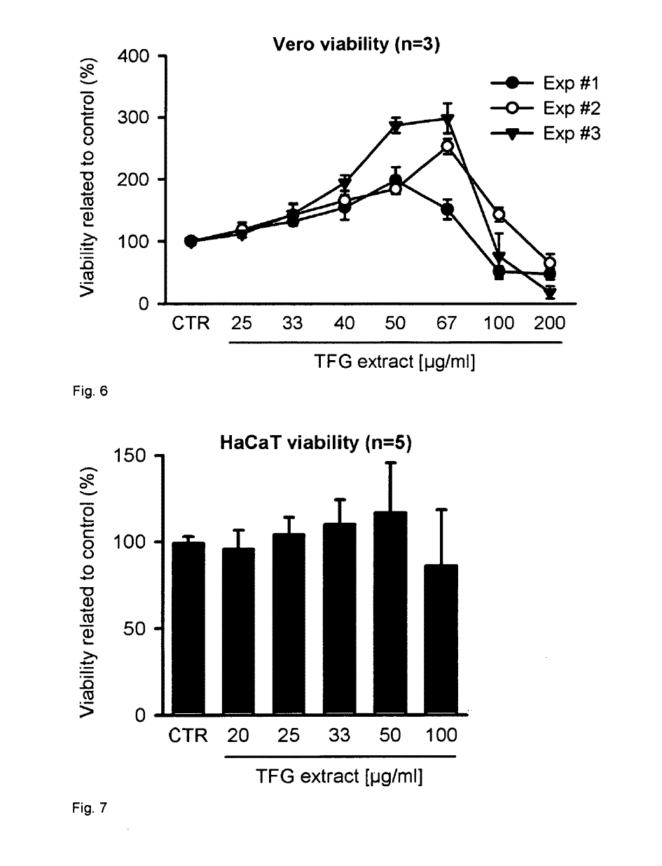

To address toxicity and effects for the extract on cell growth we added the compound in increasing concentrations to different cells cultures and human primary cells and evaluated viability after 2 days of stimulation. The TFG extract was 50% toxic concentration (TD₅₀) in the concentration range 100-200 μg/ml for Vero cells (FIG. 6), and human PBMCs (FIG. 8). The human keratinocyte cell line HaCaT had a TD₅₀>100 μg/ml (FIG. 7).

Interestingly, epithelial Vero cells showed increased proliferation in the TFG concentration range 30-70 μg/ml (FIG. 6) and a similar trend was seen for HaCaT keratinocytes with a consistent increase in cell number at a TFG extract concentration of 50 μg/ml (FIG. 7) compared to controls with no TFG extract. A similar trend may be observed in FIG. 8 for PBMC cells. In conclusion, TFG extracts are non-toxic in the full range of antiviral activity against HSV-2 (0.1-2.5 μg/ml in pre-incubation tubes, FIG. 6) and non-toxic at the upper range of anti-HIV-1 antiviral activity (10-40 μg/ml in pre-incubation tubes, FIG. 7). Moreover, the data suggest a proliferative effect at a certain concentrations of TFG extract.

Rapid Antiviral Effect Primarily Via Direct Interaction with the Virus

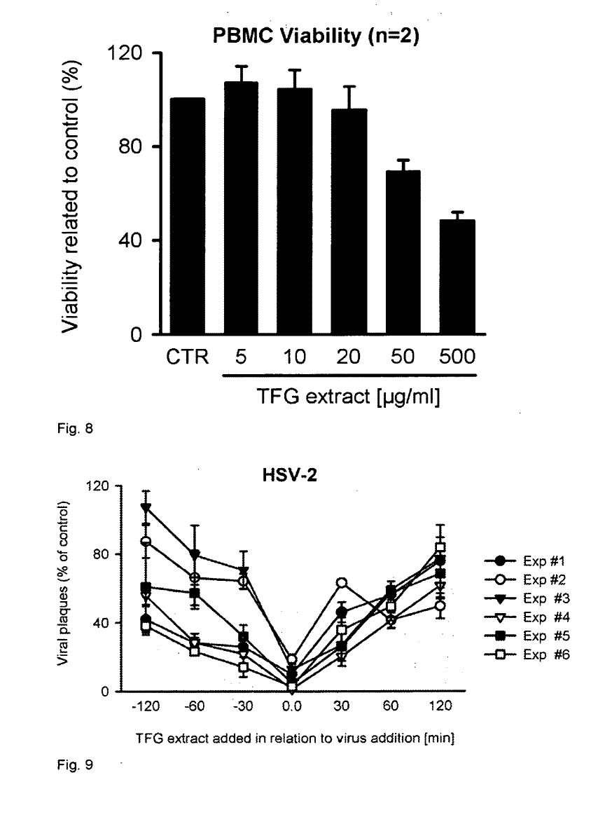

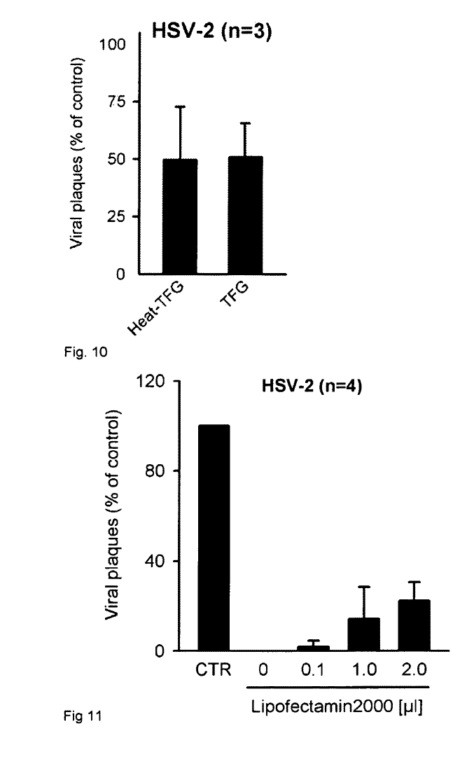

Next, we determined whether the effect observed is a direct virucidal effect or an effect at later time points in infection, including inhibition of entry and replication. We, therefore, performed a number of experiments in which we added TFG extract (20 μg/ml) ranging from 2 h before infection to 2 h after infection. The maximum antiviral effect was seen if the extract was applied at the time of infection (FIG. 9). The antiviral effect gradually decreased if the TFG extracts was added before or after the time of infection with 50-55% antiviral activity if the extract was added 1 h before or 1 h after infection and 30-35% antiviral effect if the TFG extracts was added either 2 hours before or after infection compared to controls with no extract. Knowing that the TFG extract exerted its effect most efficiently when applied together with virus, we next wished to investigate the incubation period needed for efficient antiviral effect. We incubated HSV-2 with TFG extract (1 μg/ml) for either 30 sec or 5 min before addition to Vero cells. We found that the extract acted very fast and only seconds of incubation time reduced virus levels with 15-85% and 5 min of incubation of virus and TFG extract resulted in virtually no virus infection. In conclusion, the TFG extract acts via direct interaction with the virus and the TFG extract possibly via directly interfering with the virus particle and inhibiting early steps of infection. Next, we determined the stability of the TGF extracts. We found that the anti-HSV-2 effect was intact in extracts heated to 56° C. (FIG. 10) and after weeks of storage in solution (data not shown). Collectively, the data show that TFG-extracts are very stable and suggest that the mechanism of action is via direct interaction with the virus particle and/or inhibition of early infection steps.

Antiviral Effects of TFG Plant Extract is Inhibited by Lipid Competition

Knowing that the TFG extract probably directly interacts with the virus particle and/or interferes with early steps in infection and that certain antiviral compounds interacts directly with lipid membranes on enveloped viruses, including HSV-2 and HIV-1 (Wolf et al., 2010), we investigated whether lipids would interfere with the antiviral effect observed. We found that lipid addition strongly reduced the antiviral effect (FIG. 11). In conclusion, the data suggest that TFG antiviral effect is via direct interaction with the virus membrane.

TFG Extract Mediates Increased Levels of Pro-Inflammatory IL-6 and CCL3

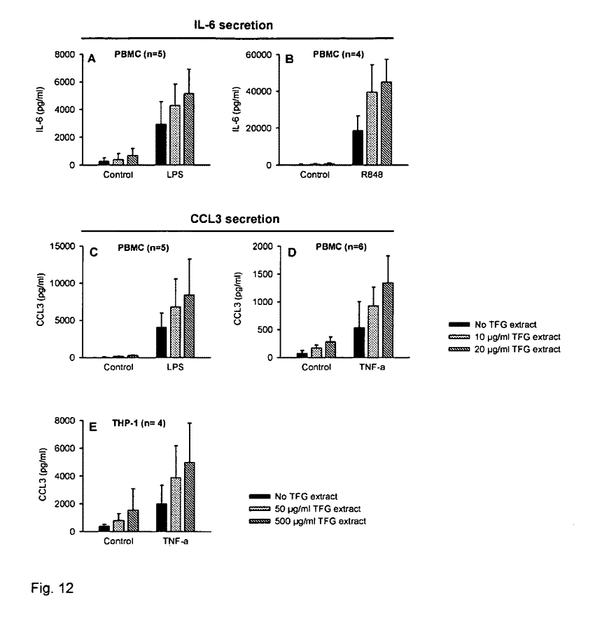

In order to evaluate the medical potential of TFG plant extracts, we subsequently investigated the effects of the extract on inflammation and innate cytokine responses in human cells. Human PBMCs were pre-treated with 10 μg/ml or 20 μg/ml TFG extract or media for 30 min before stimulation with bacterial endotoxin/lipopolysaccharide (LPS) that triggers cell surface toll-like receptor 4 (TLR4) activation and with R848, which is a ligand for endosomally located TLR7/8. In addition, we stimulated cells with the inflammatory mediator TNF-α. TFG extract induced IL-6 and CCL3/MIP-1α in human PBMCs (FIGS. 12A and 12D). Similarly, human monocytic THP-1 cells respond with CCL3 after stimulation with TFG extract (50 and 500 μg/ml) (FIG. 12E). Moreover, addition of TFG extract augmented the LPS-, R848- and TNF-α-triggered IL-6 and CCL3 responses in both PBMCs and THP-1 cells (FIG. 12A to E). In conclusion, TFG seed extract induces pro-inflammatory IL-6 and CCL3 and augments IL-6 and CCL3 production after triggering of innate pathogen sensors TLR4 and TLR7/8, as well as after TNF-α stimulation.

TFG Seed Extract Effects on IL-10 and IL-12 Secretion

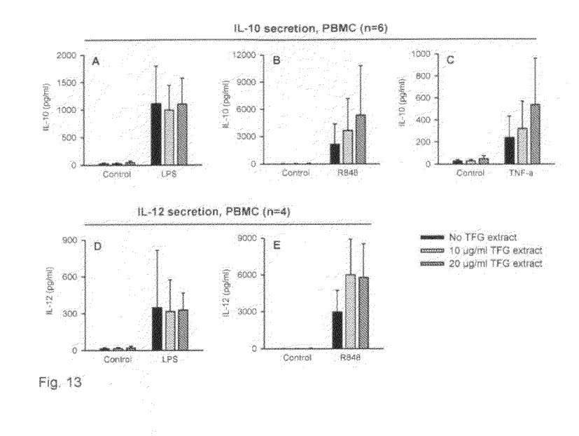

To evaluate a broader immune-modulatory effect of the TFG seed extracts, we investigated the secretion of the important regulators of inflammation and antiviral responses, IL-10 and 11-12. IL-10 is a general suppressor of inflammation and IL-12 as a key regulator of efficient anti-viral responses (Couper et al., 2008; Trinchieri, 2003; Wafford et al., 2003). PBMCs were stimulation with LPS, R848 or TNF-α in the presence or absence of TFG extracts. Neither IL-10 nor IL-12 secretion was significantly induced by TFG extract (10 and 20 μg/ml) (FIGS. 13A, C and D). Also LPS-induced IL-10 and IL-12 was not affected by the presence of TFG extract (FIGS. 13A and D). However, R848 induction of IL-10 and IL-12 was enhanced by TFG extract (FIGS. 13B and E). Similarly, TNF-α-induced IL-10 was augmented in the presence of TFG seed extract. Together, the data suggest that TFG does not induce IL-10 or IL-12 alone, but the TFG extract selectively increase levels of IL-10 and IL-12 by R848 or TNF-α stimulation.

Microbicide Containing TFG Inhibits Vaginal Infection with HSV-2

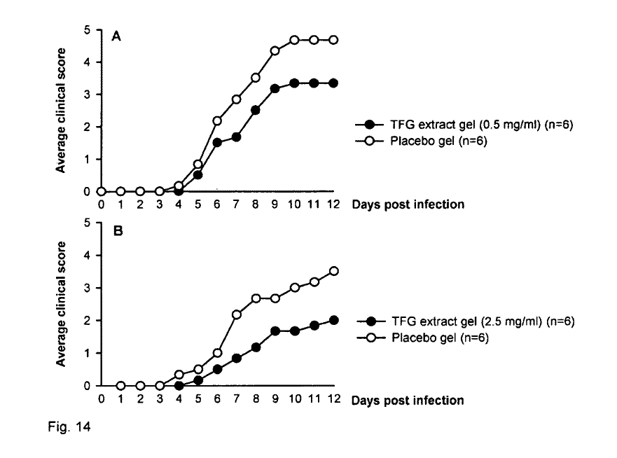

To evaluate the direct potential of TFG extracts in vivo, we used a TFG microbicide in a mouse vaginal challenge model. The mice were applied a gel containing 0.5 or 2.5 pg/ml of TFG extracts 12 h before and 12 h after HSV-2 infection. Every following day, the mice were scored using a standardized clinical score. In the experiments using 0.5 μg/ml TFG gel two of the experiments TFG gel decreased the clinical score, whereas the third did not show a significant effect. The mean of the experiments using 0.5 μg/ml TFG gels is depicted in FIG. 14A. Using a gel containing 2.5 μg/ml of TFG extract, we also found less severe disease in mice receiving the TFG-containing gel (FIG. 14B). In conclusion, the data show that TFG extracts formulated in a gel may attenuate HSV-2 infection in a vaginal challenge model.

Discussion

HSV-2 and HIV-1 are human pathogens affecting a major part of humans in the world. The viruses result in latent infections. For neither virus no cure nor vaccine is available. Furthermore, the spread of the viruses is difficult to control especially in less developed parts of the world. As a consequence alternative ways of restricting the virus infections and spread is of highest importance.

In this paper, we have shown that extracts of the seed from the legume TGF (fenugreek) possess antiviral activity against HSV-2 and HIV-1 (FIGS. 2 and 3). We found that the extracts were active against HSV-2 and HIV-1 at non-toxic concentrations (FIGS. 6 to 8) and propose that the mechanism is via direct interaction with the virus envelope (FIGS. 9 and 10). Since HSV-2 and HIV-1 are sensitive to detergents (Krebs et al., 1999; Zeitlin et al., 1997), we considered that antiviral effect to be a detergent effect e.g. via saponins. However, the anti-viral HSV-2 effect was found at very low concentrations below 100 ng/ml TFG extract in PBS, which was substantially lower than the toxic effect range seen for all cells tested. Therefore, we excluded detergent effects to be the primary cause of the anti-HSV-2 effect. However, it cannot be excluded that the anti-HIV-1 effect partly mediated by an detergent effect, since the antiviral TFG extract concentration in the pre-incubation step (Tube conc., FIG. 3) is in the range of the cytotoxic concentration found in TZM-bl cells after 2 days of incubation. A pH-dependent effect was excluded, because TFG solutions in contact with virus and cells were pH neutral and in buffered solutions.

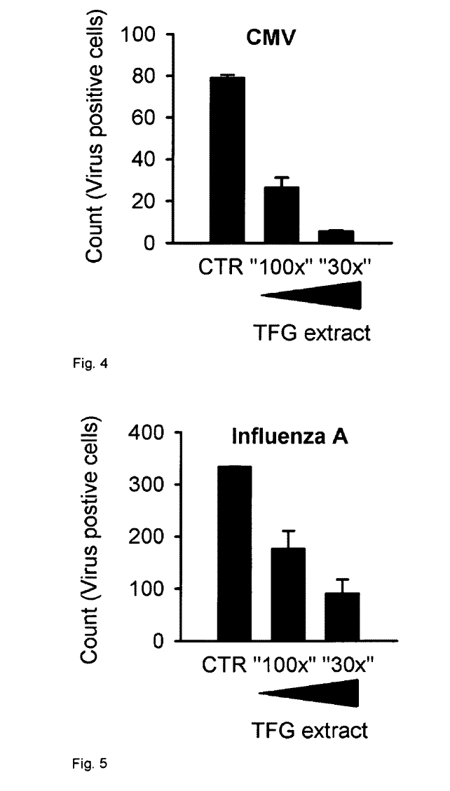

Looking at the mechanism of the antiviral effect, we found TFG extracts worked most efficiently when present at the time of infection (FIG. 9). The data suggested direct interaction with the virus, but also showed that the antiviral effect of TFG extract is persistent in cell culture, since the stability is high and antiviral effect is observed both when TFG is added 2 hours before and 2 hours after infection. Looking at the mechanism of the antiviral effect, we found that addition of lipid to the TFG extract interferes with the extracts antiviral effect (FIG. 4), thus suggesting that the antiviral compound in TFG extract binds to lipid membranes.

In addition to antiviral effects, we found that TFG extracts affect cell in several ways: i) restricting cell viability/cell growth at concentrations 100 μg/ml, ii) inducing proliferation in certain cells at specific concentration ranges and iii) modulating immune responses by inducing and augmenting cytokine responses. Specifically, we found that TFG concentrations above 100-200 μg/ml reduces cell viability or cell growth in the cells tested (FIGS. 6 to 8).

Interestingly, we also observed a cell proliferative effect in Vero cells the range 40-70 μg/ml (FIG. 6) and a similar trend was seen in human keratinocyte HaCaT cells at the TFG concentration 50 μg/ml (FIG. 7). The finding is interesting, since virus infections may lead to epithelial cell lysis and keratinocyte layer breach it is tempting to speculate that TFG extract components may have some wound healing effect in addition to the antiviral effect seen by the TFG extract. A wound healing effect would be advantageous e.g. by HSV-2 infection of the skin and mucosa. In the case of Vero cells and HaCat cells, the mechanism for TFG extract-induced increase in cell number is not found. However, one effect could be stimulation of growth factors via the estrogen receptor system, which has recently been shown for keratinocytes (Rock et al., 2012).

Immune-modulatory effects of TFG extracts were investigated in human cell cultures. TFG extract induced levels of pro-inflammatory IL-6 and CCL3, but not immune-regulatory IL-10 and IL-12. However, the presence of TFG extract enhanced secreted levels of IL-6, CCL3, IL-10 and IL-12 (FIGS. 12A to 12D and 13B, 13C and 13E), with the exception that LPS-induced levels of IL-10 and IL-12 were not affected by the presence of TFG extract (FIGS. 13A and 13B). We cannot explain why IL-10 and IL-12 is not affected by TFG extracts during LPS stimulation whereas TLR7/8 (R848) and TN F-α-induced IL-10 and IL-12 were enhanced by TFG extract. One explanation would be a slight contamination with bacterial endotoxins, which could desensitize the cells to further stimulation (Randow et al., 1995). However, we find that explanation unlikely, since CCL3 and IL-6 levels increase after LPS-stimulation also in the presence of TFG extract. Furthermore, we could not detect any LPS-response in HEK293 cells stably transfected with TLR4 (data not shown). Since PBMCs is a heterogenous cell population, another explanation is that the TFG extract affects different cells in responds to TNF-α, LPS and R848. Knowing that monocyte-like THP-1 cells respond with CCL3 similarly as PBMCs it is likely that TFG extract-mediated increase in pro-inflammatory cytokines is to some extend is mediated via TFG extract interaction with monocytes.

Bin-Hefeex et al. reported an immune-stimulatory effect of TFG extracts in mice. The immune-stimulatory effect included increased delayed hypersensitivity responses and in vitro increased macrophage phagocytotic function (Bin-Hafeez et al., 2003). Our data and the data from Bin-Hefeez et al. suggest that TFG acts partly at the innate level via the sentinel immune regulators of the myeloid lineage, including monocytes and macrophages. The collective data suggests that TFG modulation of immune functions has to be taken into account when developing new drugs or treatments. The inflammation-inducing capacity may be positive for generating local anti-microbial effects, but inflammation may also be negative to topically applied creams and gels. For instance in the case of HIV-1, microbicide-induced inflammation may be harmful and provide both activated cells for HIV-1 infection and recruitment of additional target cells to the site of application (Fichorova, 2004). Maybe the cytokine-inducing compounds should be removed from our TFG extracts before use in a vaginal cream. Our results showing an increase in inflammatory cytokines is in contrast to other studies showing a decrease in phorbol-12-myristate-13-acetate (PMA)-induced TNF-α in the presence of TFG methanol-extracts mediated by saponins (Kawabata et al., 2011). Furthermore, TFG seed extracts may interact with the endocrine system (Sreeja et al., 2010) and thus may regulate a number of estrogen receptor-regulated immune responses, including negatively affecting maturation of dendritic cells (DCs) and enhancing TLR-responses from plasmacytoid DC, (Escribese et al., 2008; Seillet et al., 2012). It remains to be determined if TFG affect general innate cytokine responses and whether the immune-modulatory has in vivo relevance.

To make a first proof of concept of the use of TFG extracts for topical application, we evaluated a microbicide gel containing TFG seed extract in a mouse HSV-2 vaginal challenge model. We found that gels containing TFG at the concentration 0.5 and 2.5 μg/ml bot had some positive effect on HSV-2 progression (FIGS. 14A and 14B). We used a lethal dose of HSV-2 which may be the reason of infected mice in the TFG group. Another reason may be the heterogenicity of the TFG extract and not knowing if some compounds in the extract are increasing HSV-2 infection in vivo, whereas others are restricting the virus. Since the mice were not completely

It should be emphasized that the content of TFG extracts may differ dependent on geography of the plant and the procedure used to make the extracts (Taylor et al., 2002). Because of thee differences in preparation and differences in phytochemical content of the seed and plant it is very difficult to extrapolate results from one study to another.

In summary, the present study provides new knowledge on TFG's antiviral, cell stimulatory and immune-regulatory effects. To our knowledge our study is the very first study showing antiviral effect of TFG extracts and how the extract may affect cytokine balances. The studies may together with the preliminary proof-of-concept studies in mice constitute the basis for future developments of antimicrobial creams and microbicides against the major human pathogens such as HSV-2 and HIV-1. Moreover, the results warrant further studies of the chemical content of TFG extracts.

### REFERENCE LIST

Ank, N., West, H., Bartholdy, C., Eriksson, K., Thomsen, A. R. & Paludan, S. R. (2006). Lambda Interferon (IFN-{lambda}), a Type III IFN, Is Induced by Viruses and IFNs and Displays Potent Antiviral Activity against Select Virus Infections In Vivo. J Virol 80, 4501-4509.
Basch, E., Ulbricht, C., Kuo, G., Szapary, P. & Smith, M. (2003). Therapeutic applications of fenugreek. Altem Med Rev 8, 20-27.
Bin-Hafeez, B., Haque, R., Parvez, S., Pandey, S., Sayeed, I. & Raisuddin, S. (2003), Immunomodulatory effects of fenugreek (Trigonella foenum graecum L.) extract in mice. Int Immunopharmacol 3, 257-265.
Couper, K. N., Blount, D. G. & Riley, E. M. (2008). IL-10: the master regulator of immunity to infection. J Immunol 180, 5771-5777.
Duke, J. A. (2001). Phytochemical constituends of GRAS herbs and other ecological plants. CRC PRess LLC.
Escribese, M. M., Kraus, T., Rhee, E., Fernandez-Sesma, A., Lopez, C. B. & Moran, T. M. (2008). Estrogen inhibits dendritic cell maturation to RNA viruses. Blood 112, 4574-4584.
Fichorova, R. N. (2004). Guiding the vaginal microbicide trials with biomarkers of inflammation. J Acquir Immune Defic Syndr 37 Suppl 3, S184-S193.
Freeman, E. E., Weiss, H. A., Glynn, J. R., Cross, P. L., Whitworth, J. A. & Hayes, R. J. (2006). Herpes simplex virus 2 infection increases HIV acquisition in men and women: systematic review and meta-analysis of longitudinal studies. AIDS 20, 73-83.
Hibasami, H., Moteki, H., Ishikawa, K., Katsuzaki, H., Imai, K., Yoshioka, K., Ishii, Y. & Komiya, T. (2003). Protodioscin isolated from fenugreek (Trigonella foenum graecum L.) induces cell death and morphological change indicative of apoptosis in leukemic cell line H-60, but not in gastric cancer cell line KATO III. Int J Mol Med 11, 23-26.
Kaviarasan, S., Ramamurty, N., Gunasekaran, P., Varalakshmi, E. & Anuradha, C. V. (2006). Fenugreek (Trigonella foenum graecum) seed extract prevents ethanol-induced toxicity and apoptosis in Chang liver cells. Alcohol Alcohol 41, 267-273.
Kawabata, T., Cui, M. Y., Hasegawa, T., Takano, F. & Ohta, T. (2011). Anti-inflammatory and anti-melanogenic steroidal saponin glycosides from Fenugreek (Trigonella foenum-graecum L.) seeds. Planta Med 77, 705-710.
Kirkegaard, T., Wheatley, A., Melchjorsen, J., Bahrami, S., Pedersen, F. S., Center, R. J., Purcell, D. F., Ostergaard, L., Duch, M. & Tolstrup, M. (2011). Induction of humoral and cellular immune responses against the HIV-1 envelope protein using gamma-retroviral virus-like particles. Virol J 8, 381.
Krebs, F. C., Miller, S. R., Malamud, D., Howett, M. K. & Wigdahl, B. (1999). Inactivation of human immunodeficiency virus type 1 by nonoxynol-9, C31G, or an alkyl sulfate, sodium dodecyl sulfate. Antiviral Res 43, 157-173.
Looker, K. J., Garnett, G. P. & Schmid, G. P. (2008). An estimate of the global prevalence and incidence of herpes simplex virus type 2 infection. Bull World Health Organ 86, 805-12, A.
Palaniswamy, M., Pradeep, B. V., Sathya, R. & Angayarkanni, J. (2010). In Vitro Anti-plasmodial activity of Trigonella foenum-graecum L. Evid Based Complement Alternat Med 7, 441-445.
Randhir, R., Lin, Y. T. & Shetty, K. (2004). Phenolics, their antioxidant and antimicrobial activity in dark germinated fenugreek sprouts in response to peptide and phytochemical elicitors. Asia Pac J Clin Nutr 13, 295-307.
Randhir, R. & Shetty, K. (2007). Improved alpha-amylase and Helicobacter pylori inhibition by fenugreek extracts derived via solid-state bioconversion using Rhizopus oligosporus. Asia Pac J Clin Nutr 16, 382-392.
Randow, F., Syrbe, U., Meisel, C., Krausch, D., Zuckermann, H., Platzer, C. & Volk, H. D. (1995). Mechanism of endotoxin desensitization: involvement of interleukin 10 and transforming growth factor beta. J Exp Med 181, 1887-1892.
Rebbapragada, A., Wachihi, C., Pettengell, C., Sunderji, S., Huibner, S., Jaoko, W., Ball, B., Fowke, K., Mazzulli, T., Plummer, F. A. & Kaul, R. (2007). Negative mucosal synergy between Herpes simplex type 2 and HIV in the female genital tract. AIDS 21, 589-598.
Rock, K., Meusch, M., Fuchs, N., Tigges, J., Zipper, P., Fritsche, E., Krutmann, J., Homey, B., Reifenberger, J. & Fischer, J. W. (2012). Estradiol Protects Dermal Hyaluronan/Versican Matrix during Photoaging by Release of Epidermal Growth Factor from Keratinocytes. J Biol Chem 287, 20056-20069.
Roizman, B., Knipe, D. M. & Whitley, R. J. (2007). Herpes simplex virus. In Fields Virology, 5 edn, pp. 2501-2601. Edited by D. M. Knipe, P. M. Howley, D. E. Griffin, R. A. Lamb, M. A. Martin, B. Roizman & S. E. Straus: Lippincott Williams & Wilkins.
Seillet, C., Laffont, S., Tremollieres, F., Rouquie, N., Ribot, C., Arnal, J. F., Douin-Echinard, V., Gourdy, P. & Guery, J. C. (2012). The TLR-mediated response of plasmacytoid dendritic cells is positively regulated by estradiol in vivo through cell-intrinsic estrogen receptor alpha signaling. Blood 119, 454-464.
Shabbeer, S., Sobolewski, M., Anchoori, R. K., Kachhap, S., Hidalgo, M., Jimeno, A., Davidson, N., Carducci, M. A. & Khan, S. R. (2009). Fenugreek: a naturally occurring edible spice as an anticancer agent. Cancer Biol Ther 8, 272-278.
Skaltsa, H. (2002). Chemical constituents. In Fenugreek—the genus Trigonella, pp. 132-161. Edited by G. A. Petropoulos: Taylor & Francis.
Sreeja, S., Anju, V. S. & Sreeja, S. (2010). In vitro estrogenic activities of fenugreek Trigonella foenum graecum seeds. Indian J Med Res 131, 814-819.
Taylor, W. G., Zulyniak, H. J., Richards, K. W., Acharya, S. N., Bittman, S. & Elder, J. L. (2002). Variation in diosgenin levels among 10 accessions of fenugreek seeds produced in western Canada. J Agric Food Chem 50, 5994-5997.
Trinchieri, G. (2003), Interleukin-12 and the regulation of innate resistance and adaptive immunity. Nat Rev Immunol 3, 133-146.
Ulbricht, C., Basch, E., Burke, D., Cheung, L., Ernst, E., Giese, N., Foppa, I., Hammerness, P., Hashmi, S., Kuo, G., Miranda, M., Mukherjee, S., Smith, M., Sollars, D., Tanguay-Colucci, S., Vijayan, N. & Weissner, W. (2007). Fenugreek (Trigonella foenum-graecum L. Leguminosae): an evidence-based systematic review by the natural standard research collaboration. J Herb Pharmacother 7, 143-177.
UNAIDS (2010). UNAIDS report on the global AIDS epidemic 2010.
Watford, W. T., Moriguchi, M., Morinobu, A. & O'Shea, J. J. (2003). The biology of IL-12: coordinating innate and adaptive immune responses. Cytokine Growth Factor Rev 14, 361-368.
Wolf, M. C., Freiberg, A. N., Zhang, T., Akyol-Ataman, Z., Grock, A., Hong, P. W., Li, J., Watson, N. F., Fang, A. Q., Aguilar, H. C., Porotto, M., Honko, A. N., Damoiseaux, R., Miller, J. P., Woodson, S. E., Chantasirivisal, S., Fontanes, V., Negrete, O. A., Krogstad, P., Dasgupta, A., Moscona, A., Hensley, L. E., Whelan, S. P., Faull, K. F., Holbrook, M. R., Jung, M. E. & Lee, B. (2010). A broad-spectrum antiviral targeting entry of enveloped viruses. Proc Natl Acad Sci USA 107, 3157-3162.
Zeitlin, L., Whaley, K. J., Hegarty, T. A., Moench, T. R. & Cone, R. A. (1997). Tests of vaginal microbicides in the mouse genital herpes model. Contraception 56, 329-335.

### Example 5

Inhibition of Plasmodium falciparum Proliferation by Extract

In vitro activity of extract dilutions were tested against erythrocytic stages of P. falciparum by a modified [3H]-hypoxanthine incorporation assay (Scala, F., Fattorusso, E., Menna, M., Taglialatela-Scafati, O., Tiemey, M., Kaiser, M., Tasdemir, D., 2010. Bromopyrrole alkaloids as lead compounds against protozoan parasites. Marine Drugs 8, 2162-2174.) using the amodiaquine sensitive strain K1. The standard drug used as a positive control was amodiaquine.

Briefly, parasite cultures incubated in RPMI 1640 medium with 5% Albumax (without hypoxanthine) were exposed to serial extract dilutions in microtiter plates. After 48 h of incubation at 37 ° C. in a reduced oxygen atmosphere, 0.5 μCi 3H-hypoxanthine was added to each well. Cultures were incubated for a further 24 h before they were harvested onto glass-fiber filters and washed with distilled water. The radioactivity was counted using a Betaplate™ liquid scintillation counter (Wallac, Zurich, Switzerland). The results were recorded as counts per minute (CPM) per well at each drug concentration and expressed as percentage of the untreated controls. IC50 values were calculated from graphically plotted dose-response curves. Each IC50 value obtained is the mean of at least two separate experiments performed in duplicate (the variation is maximum 20%).

The result shows that at a dilution of 160 times the extract obtains a value of 51.5 CPM, wherein the reference drug (amodiaquine) shows an activity of 47.5 CPM. Thus, the extract shows an anti-plasmodial activity similar to the activity of the reference drug.

### Example 6

Preparation of 2-methyl-3-nonyloxiran-2-amine

2-Nitro-dodecan-3-ol.

58.1 g (0.37 mol) Decanal (0.37 mol); 55.8 g (0.74 mol) nitroethane and 1.75 g (19 mol) Potassium fluoride was mixed with 400 mL 2-propanol and stirred at room temperature for 48 hours. Anhydrous MgSO₄ was added, the mixture filtered and concentrated in vacuo. Yield 80.8 g (94%).

NMR according to the literature data (D. L. Haire, E. G . Janzen: Can. J. Chem. 60, 1514 (1982))

2-Nitrododecan-3-yl acetate.

2.4 g (10.4 mmol) 2-Nitro-dodecan-3-ol was added with stirring to 1.15 (11.3 mmol) Acetic anhydride precooled to 0° C. 1 drop of conc. Sulfuric acid was added and stirring continued for further 3 hours. The mixture was poured into water and extracted with diethyl ether. The organic phase was washed with NaHCO₃-solution dried over Na₂SO₄ and concentrated in vacuo to give 1.77 g (62%).

NMR according to the literature data (D. L. Haire, E. G . Janzen: Can. J. Chem. 60, 1514 (1982))

(E/Z)-2-Nitrododec-2-ene.

2.70 g (10 mmol) 2-Nitrododecan-3-yl acetate, 75 mL tert-Butanol and 1.6 g (12 mmol) was stirred at 35 oC for 10 hours, poured into water and extracted with diethyl ether. The organic phase was dried over Na₂SO₄, filtered, concentrated in vacuo and purified by dry column chromatography on Silicagel 60 using an ethyl acetate/hexane gradient. Yield: 1.2 (56%). NMR according to literature data (N. Ono, K. Maruyama: Bull. Chem. Soc. Jpn. 61, 4470-4472 (1988)).

2-Methyl-2-nitro-3-nonyloxirane.

1.87 g (8.80 mmol) (E/Z)-2-Nitrododec-2-ene was dissolved in 40 mL Methanol and cooled to 0° C. A mixture of 5 mL 30% H₂O₂ and 7.5 mL 2 M NaOH was added slowly under vigorous stirring, which was continued for 1 hour. The mixture was poured into cold 1 M HCl and extracted with diethyl ether. The organic phase was dried over Na₂SO₄, filtered and concentrated in vacuo to give 1.68 g (83%) of the product.

1H-NMR (500 MHz, CDCl3): δ 3.45 (t, 1H); 1.95 (s, 3H); 1.59 (m, 4H); 1.37 (m, 12H); 0.89 (t, 3H). 13C-NMR (125 MHz, CDCl3): δ 87.96; 63.10; 33.87; 31.85; 29.39; 29.35; 29.23; 29.12; 29.05; 28.91; 24.68; 22.64; 14.08; 13.67.

2-Methyl-3-nonyloxiran-2-amine.

0.69 g (3 mmol) 2-Methyl-2-nitro-3-nonyloxirane in 30 mL Hexane was added to a mixture of 6 g Al₂O₃ containing 19% H₂O, 10 mg CoCl₂.6H₂O and 0.23 g NaBH₄. This mixture was stirred at 30° C. for 1 hour, filtered, washed with diethyl ether and concentrated in vacuo. Yield: 0.60 g (quant).

1H-NMR (500 MHz, CDCl3): δ 3.36 (dd, 1H); 1.86 (s, 2H); 1.49 (m, 4H); 1.37 (m, 12H); 0.81 (t, 3H). 13C-NMR (125 MHz, CDCl3): δ 87.97; 63.11; 31.84; 29.40; 29.36; 29.24; 29.14; 27.88; 25.80; 22.66; 14.10; 13.71.

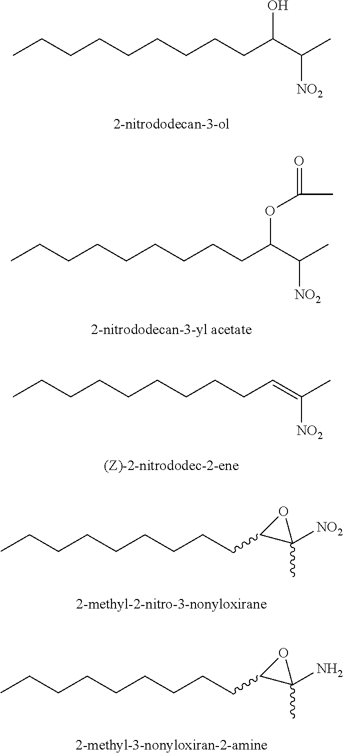

### Example 7

Inhibition of Plasmodium falciparum Proliferation by 2-Methyl-3-nonyloxiran-2-amine

15.3 mg 2-Methyl-3-nonyloxiran-2-amine obtained in example 6 was mixed with 100 μl DMSO (Dimethyl sulfoxid) to obtain a stock solution. A first solution was prepared by mixing 100 uL of the stock solution with 400 uL of parasite media. Subsequently this solution was diluted in 15 dilution series, each dilution serie being diluted 3 fold. The same protecol as used in example 6 was applied for this experiment.

Specifically, the incorporation of [3H]-hypoxanthine was used as a measure for the life conditions of P. falciparum. Parasites were syncronised to ring stages by trizol treatment and then incubated at 0.3% parasitemia at 5% haematocrit in 100 uL of growth medium containing [3H]-hypoxanthine over one cycle of replication, i.e. 48 hrs. Each experiment was performed in triplets.

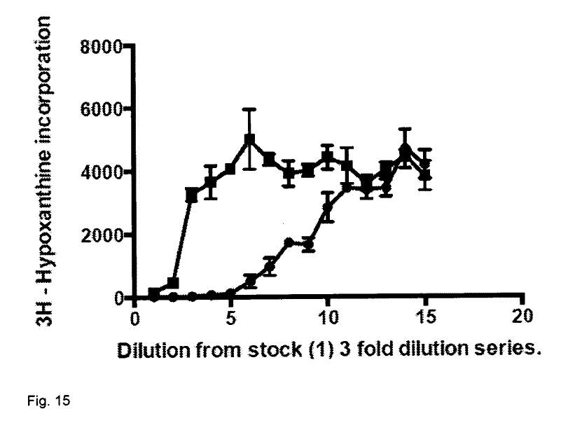

The result of the experiment is shown in FIG. 15, which shows a concentration dependent inhibition of the growth rate of P. Falciparum in the presence of 2-Methyl-3-nonyloxiran-2-amine.

### Example 8

Effect of 2-Methyl-3-nonyloxiran-2-amine on MRSA og MSSA

15.3 mg 2-Methyl-3-nonyloxiran-2-amine obtained in example 6 was mixed with 100 μl DMSO (Dimethyl sulfoxid) to obtain a stock solution. The stock solution was used for determining the MIC (Minimum Inhibitory Concentration) for MRSA (meticillin-resistant staphylococcus aureusis) and MSSA (meticillin-sensitive staphylococcus aureusis).

The MRSA and the MSSA values were measured in dilition series to be 38.3 mg/ml and 9.6 mg/ml, respectively. Thus, the tested compound has a anti-microbial towards the two tested micro-organisms.

1. A compound of the general formula: 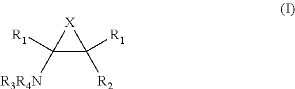
wherein
X represents O or S,
R₁ independently represents hydrogen; a straight or branched alkyl, alkenyl or alkynyl group containing up to 6 carbon atoms, optionally substituted by one or more halogen atoms or one or more groups R⁵; or a cycloalkyl or cycloalken group containing from 3 to 7 carbon atoms, said group optionally being substituted by one or more groups R⁵ or one or more halogen atoms,
R₂ represents a straight or branched alkyl, alkenyl or alkynyl group containing 3 to 24 carbon atoms, said group optionally being substituted by one or more halogen atoms, a cycloalkyl group containing from 3 to 6 carbon atoms, or one or more groups R⁵,
R₃ and R₄ may independently represent hydrogen, a straight or branched alkyl, alkenyl or alkynyl group containing up to six carbon atoms, said group optionally being substituted by one or more halogen atoms, or may together with the nitrogen atom to which they are joined or together with R₁ form 5 to 7 membered saturated or unsaturated heterocyclic ring containing up to three ring heteroatoms selected from nitrogen, oxygen and sulfur, which ring is optionally substituted by one or more groups selected from halogen, nitro, —S(O)pR⁶, C₁₋₄ alkyl, C₁₋₄ alkoxy, C₁₋₄ haloalkyl, C₁₋₄ haloalkoxy, ═O, and ═NO-R⁵, it being understood that a sulphur atom, where present in the ring, may be in the form of a group —SO₂— or —SO—;
R₅ represents a straight or branched alkyl group containing up to six carbon atoms, said group optionally being substituted by one or more halogen atoms, a C₁₋₄ alkoxy, or a straight or branched alkenyl or alkynyl group containing from 2 to 6 carbon atoms, said group optionally being substituted by one or more halogen atoms; and
R⁶ represents a straight or branched alkyl group containing up to six carbon atoms, said group optionally being substituted by one or more halogen atoms;
p is 0, 1, or 3
and pharmaceutically acceptable salts thereof.

2. The compound according to claim 1, wherein R₂ represents a straight or branched alkyl or alkenyl group containing 5 or more carbon atoms.

3. The compound according to claim 1, wherein R₂ represents a straight or branched alkenyl group having 1 to 4 double bonds.

4. The compound according to claim 3, wherein a double bond is located at the third, sixth, or nineth carbon atom counted from the methyl end of the alkenyl group.

5. The compound according to claim 1, having the formula

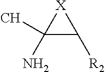

6. The compound according to claim 5, wherein R₂ is

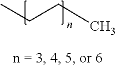

7. The composition according to claim 1, comprising the compound according to any of the claims 1 to 6 and pharmaceutically acceptable auxiliaries.

8. The compound according to claim 1, for use in a method for treatment of the human or animal body by therapy.

9. The compound according to claim 1, for use in prevention or treatment of a viral infection.

10. The compound according to claim 1, wherein said virus is selected from the group of viruses having a lipid membrane.

11. The compound according to claim 1, wherein the virus is herpes simplex virus (HSV), influenza virus, human papilloma virus (HPV) or human immunodeficiency virus (HIV).

12. The compound according to claim 1 for use in proliferating cells.

13. The compound according to claim 1, wherein it is used for treatment of wounds, such as a surgical wound or burn, diseases in the mouth cavity, periodontal disease, infection in the eye or the adnexa of the eye, cold sores, and pharyngitis.

14. The compound according to claim 13, wherein said periodontal disease is selected among gingivitis, periodontitis, and halitosis,

15. The compound according to claim 1, for use in treating diseases sensitive to cytokins.

16. The compound according to claim 15, wherein the cytokin is selected from the group comprising IL-6, CCL-3 and IL-10.

17. The compound according to claim 15, wherein the disease sensitive to cytokins is diabetes, artherosclerosis, depression, Alzheimers Disease, systemic lupus erythematosus, rheumatoid arthritis, autoimmune diseases, chronic inflammatory proliferative disease, coronary diesease, hematological and solid malignancies, asthma, arthritis, pneumonia, psoriasis or multiple sclerosis.

18. The compound according to claim 1, for use in the prevention or treatment of malaria.

19. The compound according to claim 1, for use in the prevention or treatment of infections caused by Methicillin-resistant Staphylococcus aureus (MRSA) or Methicillin-sensitive Staphylococcus aureus (MSSA).

20. The compound according to claim 1, for preparing a pharmaceutical composition.

21. The pharmaceutical composition comprising a compound according to claim 1 wherein said pharmaceutical composition is formulated as a gel, cream, mouth-wash, chewing gum, tooth-paste, balm, plaster, lip salve, spray, ointment, capsule, drop, or tablet.

22. The method for the treatment or prevention of a viral or Plasmodium infection, wherein a person suffering from a disease caused by a virus having a lipid membrane or Plasmodium is administrated an amount of a pharmaceutical composition comprising the compound according to claim 1, in an amount sufficient to cure or alleviate the disease.
